(19) United States
(12) Patent Application Publication
(10) Pub. No.: US 2024/0363220 A1
(43) Pub. Date: Oct. 31, 2024

(54) SYSTEM AND METHOD FOR GENERATING A DIRECTION INQUIRY RESPONSE FROM BIOLOGICAL EXTRACTIONS USING MACHINE LEARNING

(71) Applicant: KPN INNOVATIONS LLC, LAKEWOOD, CO (US)

(72) Inventor: Kenneth Neumann, LAKEWOOD, CO (US)

(73) Assignee: KPN INNOVATIONS LLC, LAKEWOOD, CO (US)

(21) Appl. No.: 18/765,921

(22) Filed: Jul. 8, 2024

Related U.S. Application Data
(63) Continuation-in-part of application No. 17/032,115, filed on Sep. 25, 2020, now Pat. No. 12,039,420.

(51) Int. Cl. G16H 20/60; G06F 40/279; G16H 10/60

(52) U.S. Cl. CPC G16H 20/60; G06F 40/279; G16H 10/60

(57) ABSTRACT

A system generating a directional response is disclosed. The system comprises a computing device configured to receive a directional inquiry from a device operated by a user. Computing device is configured to retrieve a biological extraction from the user and generate a directional response by training a machine-learning process using directional training data correlating a plurality of biological extractions to a plurality of directions and generating the directional response as a function of the biological extraction from the user and the machine-learning process. Computing device is configured to update the directional response as a function of the preferences of the use and output the updated directional response to the device operated by the user. A method for generating a directional response is also disclosed.

### CROSS-REFERENCE TO RELATED APPLICATIONS

This application is a continuation-in-part of Non-provisional Application Ser. No. 17/032,115, filed on Sep. 25, 2020, now U.S. Pat. No. 12,039,420, issued on Jul. 16, 2024, and entitled “SYSTEM AND METHOD FOR GENERATING A DIRECTION INQUIRY RESPONSE FROM BIOLOGICAL EXTRACTIONS USING MACHINE LEARNING,” the entirety of which is incorporated herein by reference.

### FIELD OF THE INVENTION

The present invention generally relates to the field of machine learning. In particular, the present invention is directed to systems and methods for generating directional responses from a biological extraction using machine learning.

### BACKGROUND

Efficient routing of inquiries to responses regarding directional requests remains elusive, because of the divergent criteria according to which such routing may be determined; data complexity can obscure algorithmic techniques. A resulting lack of specificity may end in dissatisfaction with resulting outputs.

### SUMMARY OF THE DISCLOSURE

In an aspect, a system for generating a directional response using machine learning includes a computing device configured to receive user data, retrieve a biological extraction of a user, generate a nutrient program as a function of the vocation data, wherein generating the nutrient program includes generating program training data, wherein the program training data includes correlations between exemplary user data, exemplary biological extractions and exemplary nutrient programs, training a program machine-learning model using the program training data and generating the nutrient program using the trained program machine-learning model, generate a directional response as a function of the nutrient program and output the directional response.

In another aspect, a method for generating a directional response using machine learning is disclosed. The method includes receiving, using a computing device, user data, retrieving, using the computing device, a biological extraction of a user, generating, using the computing device, a nutrient program as a function of the vocation data, wherein generating the nutrient program includes generating program training data, wherein the program training data includes correlations between exemplary user data, exemplary biological extractions and exemplary nutrient programs training a program machine-learning model using the program training data and generating the nutrient program using the trained program machine-learning model, generating, using the computing device, a directional response as a function of the nutrient program and outputting, using the computing device, the directional response.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram of illustrating an exemplary embodiment of the system for generating a directional response;

FIG. 2 is a block diagram illustrating an exemplary embodiment of a user database;

FIG. 3 is a block diagram of an exemplary embodiment of a machine-learning module;

FIG. 4 is a block diagram of an exemplary embodiment of a machine-learning module for generating a directional response;

FIG. 5 is a flow diagram illustrating an exemplary embodiment of a method of generating directional response;

FIG. 6 illustrates a diagram of an exemplary neural network;

FIG. 7 illustrates a block diagram of an exemplary node in a neural network;

FIG. 8 illustrates a flow diagram of an exemplary method for generating a directional response; and

FIG. 9 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to systems and methods for generating a direction inquiry response based on a biological extraction, using machine learning, where machine-learning models therefor may be generated, using machine-learning processes operating on training examples. A directional inquiry is received from a user along with a biological extraction. A trained machine-learning process is used to generate a directional response which is updated based on the user's preference. Machine-learning processes may include, without limitation, classification, regression, and/or neural network processes, which may perform unsupervised or supervised machine-learning procedures including naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. The directional response is then sent to the user-client device.

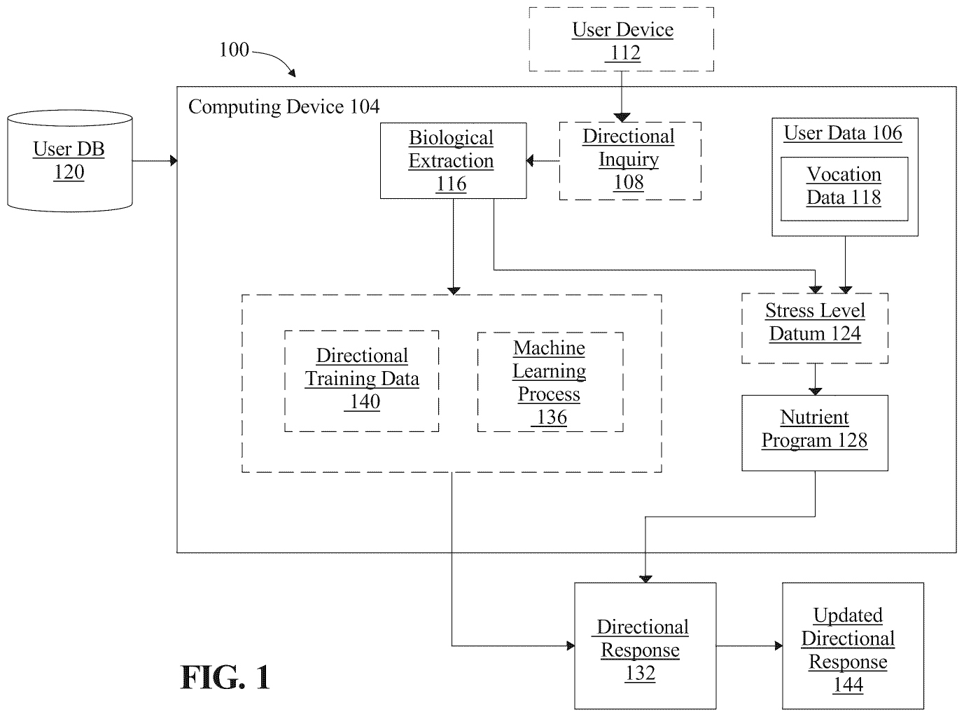

Referring now to FIG. 1, an exemplary embodiment of a system 100 for generating a direction inquiry response is illustrated. System includes a computing device 104. Computing device 104 may include any computing device as described in this disclosure, including without limitation a microcontroller, microprocessor, digital signal processor (DSP) and/or system on a chip (SoC) as described in this disclosure. Computing device may include, be included in, and/or communicate with a mobile device such as a mobile telephone or smartphone. Computing device 104 may include a single computing device operating independently, or may include two or more computing device operating in concert, in parallel, sequentially or the like; two or more computing devices may be included together in a single computing device or in two or more computing devices. Computing device 104 may interface or communicate with one or more additional devices as described below in further detail via a network interface device. Network interface device may be utilized for connecting computing device 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Computing device 104 may include but is not limited to, for example, a computing device or cluster of computing devices in a first location and a second computing device or cluster of computing devices in a second location. Computing device 104 may include one or more computing devices dedicated to data storage, security, distribution of traffic for load balancing, and the like. Computing device 104 may distribute one or more computing tasks as described below across a plurality of computing devices of computing device, which may operate in parallel, in series, redundantly, or in any other manner used for distribution of tasks or memory between computing devices. Computing device 104 may be implemented using a “shared nothing” architecture in which data is cached at the worker, in an embodiment, this may enable scalability of system 100 and/or computing device.

Still referring to FIG. 1, computing device 104 may be designed and/or configured to perform any method, method step, or sequence of method steps in any embodiment described in this disclosure, in any order and with any degree of repetition. For instance, computing device 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Still referring to FIG. 1, computing device 104 is configured to user data 106. For the purposes of this disclosure, “user data” is data related to a user. In some embodiments, computing device 104 may receive user data 106 from directional inquiry 108 described below. In some embodiments, user data 106 may be received from user device 112. As a non-limiting example, user data 106 may include user's demographic, medical history, and the like. As another non-limiting example, user data 106 may include information related to a family history of the user related to biological extraction 116; for example, and without limitation, family medical history such as chronic diseases, mental health disorders, cancers, genetic disorders, and the like. For the purposes of this disclosure, a “family history” is information about a user's family. User data 106 includes vocation data 118. For the purposes of this disclosure, “vocation data” is data related to a vocation of a user. For the purposes of this disclosure, a “vocation” is a specific position of employment or field of employment. As a non-limiting example, vocation data 118 may include a user's job, responsibility, work experience, career span, and the like. In some embodiments, user may manually input user data 106 and vocation data 118. In some embodiments, computing device 104 may retrieve user data 106 and vocation data 118 from database.

Still referring to FIG. 1, computing device 104 may receive a direction inquiry 108 from a device of user. As used in this specification, a “directional inquiry” is defined as a request for advice about a career, a job, or an area of interest where a user may be drawn, or for which they are suited, trained, or qualified; a direction inquiry may correspond to a direction in which a person may take a career or the like. A directional inquiry 108 may include an interest on a paid position, a voluntary position, an internship, a fellowship, and the like. A directional inquiry 108 may include an interest on a full time or a part time position. For example, a user may find that they are drawn to working with automobiles. After entering such an interest in the user-client device, the user may receive a directional response that may match the user with a career as a mechanic. As another example, a similar interest in automobiles may result in a directional response of a direction as a car salesperson. As used in this disclosure, a “directional response” is a match made between a directional inquiry and a user related to a vocation of the user. A directional inquiry 108 may include an interest in a religious life. For example, an interest in a religious direction may generate a direction such as becoming a Roman Catholic priest, a Rabbi, an Iman, a Nun, or other possible directions that involve religion. A directional inquiry 108 may be received from a user device. As used in this disclosure, a “device of user” is any device capable of supporting a request from a user in creating a directional inquiry 108. User device 112 may include any type of telephonic device, such as, but not limited to a mobile phone, a smartphone, a telephone connected to a local area network line, and the like. User device 112 may include other devices capable such as, but not limited to a laptop computer, a tablet computer, a desktop computer, and the like. User device 112 may include voice-controlled intelligent personal assistance modules, or the like. Further examples of user device 112 will be apparent to persons of skill in the art upon reading the entirety of this disclosure, and may include, without limitation, an interactive chatterbot configured in, for example, a smartphone to interact with the user.

Still referring to FIG. 1, computing device 104 is configured to retrieve a biological extraction 116 from the user. A “biological extraction” as used in this disclosure is an element of data including at least an element of physiological state data of a user. As used in this disclosure, “physiological state data” is any data indicative of a person's physiological state; physiological state may be evaluated with regard to one or more measures of health of a person's body, one or more systems within a person's body such as a circulatory system, a digestive system, a nervous system, or the like, one or more organs within a person's body, and/or any other subdivision of a person's body useful for diagnostic or prognostic purposes. For instance, and without limitation, a particular set of biomarkers, test results, and/or biochemical information may be recognized in a given medical field as useful for identifying various disease conditions or prognoses within a relevant field. As a non-limiting example, and without limitation, physiological state data describing red blood cells, such as red blood cell count, hemoglobin levels, hematocrit, mean corpuscular volume, mean corpuscular hemoglobin, and/or mean corpuscular hemoglobin concentration may be recognized as useful for identifying various conditions such as dehydration, high testosterone, nutrient deficiencies, kidney dysfunction, chronic inflammation, anemia, and/or blood loss. As another non-limiting example, physiological state data or biological extraction 116 may include information related to hydration, muscles and metabolism, body fats and angry fats, bone support, blood pressure, emotional impactors, cellular energy, heart or vascular, immunity, diabetic indicators, metabolic syndrome indicators, digestion, liver, inflammation, molecular rusting, and the like and computing device 104 may analyze physiological state data or biological extraction listed in this disclosure. In some embodiments, physiological state data may include health history information, pharmaceutical drug history, family health history, and the like.

Still referring to FIG. 1, physiological state data may include, without limitation, hematological data, such as red blood cell count, which may include a total number of red blood cells in a person's blood and/or in a blood sample, hemoglobin levels, hematocrit representing a percentage of blood in a person and/or sample that is composed of red blood cells, mean corpuscular volume, which may be an estimate of the average red blood cell size, mean corpuscular hemoglobin, which may measure average weight of hemoglobin per red blood cell, mean corpuscular hemoglobin concentration, which may measure an average concentration of hemoglobin in red blood cells, platelet count, mean platelet volume which may measure the average size of platelets, red blood cell distribution width, which measures variation in red blood cell size, absolute neutrophils, which measures the number of neutrophil white blood cells, absolute quantities of lymphocytes such as B-cells, T-cells, Natural Killer Cells, and the like, absolute numbers of monocytes including macrophage precursors, absolute numbers of eosinophils, and/or absolute counts of basophils. Physiological state data may include, without limitation, immune function data such as Interleukine-6 (IL-6), TNF-alpha, systemic inflammatory cytokines, and the like.

Continuing to refer to FIG. 1, physiological state data may include, without limitation, data describing blood-born lipids, including total cholesterol levels, high-density lipoprotein (HDL) cholesterol levels, low-density lipoprotein (LDL) cholesterol levels, very low-density lipoprotein (VLDL) cholesterol levels, levels of triglycerides, and/or any other quantity of any blood-born lipid or lipid-containing substance. Physiological state data may include measures of glucose metabolism such as fasting glucose levels and/or hemoglobin A1-C (HbA1c) levels. Physiological state data may include, without limitation, one or more measures associated with endocrine function, such as without limitation, quantities of dehydroepiandrosterone (DHEAS), DHEA-Sulfate, quantities of cortisol, ratio of DHEAS to cortisol, quantities of testosterone quantities of estrogen, quantities of growth hormone (GH), insulin-like growth factor 1 (IGF-1), quantities of adipokines such as adiponectin, leptin, and/or ghrelin, quantities of somatostatin, progesterone, or the like. Physiological state data may include measures of estimated glomerular filtration rate (eGFR). Physiological state data may include quantities of C-reactive protein, estradiol, ferritin, folate, homocysteine, prostate-specific Ag, thyroid-stimulating hormone, vitamin D, 25 hydroxy, blood urea nitrogen, creatinine, sodium, potassium, chloride, carbon dioxide, uric acid, albumin, globulin, calcium, phosphorus, alkaline phosphatase, alanine amino transferase, aspartate amino transferase, lactate dehydrogenase (LDH), bilirubin, gamma-glutamyl transferase (GGT), iron, and/or total iron binding capacity (TIBC), or the like. Physiological state data may include antinuclear antibody levels. Physiological state data may include aluminum levels. Physiological state data may include arsenic levels. Physiological state data may include levels of fibrinogen, plasma cystatin C, and/or brain natriuretic peptide.

Continuing to refer to FIG. 1, physiological state data may include measures of lung function such as forced expiratory volume, one second (FEV-1) which measures how much air can be exhaled in one second following a deep inhalation, forced vital capacity (FVC), which measures the volume of air that may be contained in the lungs. Physiological state data may include a measurement blood pressure, including without limitation systolic and diastolic blood pressure. Physiological state data may include a measure of waist circumference. Physiological state data may include body mass index (BMI). Physiological state data may include one or more measures of bone mass and/or density such as dual-energy x-ray absorptiometry. Physiological state data may include one or more measures of muscle mass. Physiological state data may include one or more measures of physical capability such as without limitation measures of grip strength, evaluations of standing balance, evaluations of gait speed, pegboard tests, timed up and go tests, and/or chair rising tests.

Still viewing FIG. 1, physiological state data may include one or more measures of cognitive function, including without limitation Rey auditory verbal learning test results, California verbal learning test results, NIH toolbox picture sequence memory test, Digital symbol coding evaluations, and/or Verbal fluency evaluations. Physiological state data may include one or more evaluations of sensory ability, including measures of audition, vision, olfaction, gustation, vestibular function and pain.

Continuing to refer to FIG. 1, physiological state data may include psychological data. Psychological data may include any data generated using psychological, neuro-psychological, and/or cognitive evaluations, as well as diagnostic screening tests, personality tests, personal compatibility tests, or the like; such data may include, without limitation, numerical score data entered by an evaluating professional and/or by a subject performing a self-test such as a computerized questionnaire. Psychological data may include textual, video, or image data describing testing, analysis, and/or conclusions entered by a medical professional such as without limitation a psychologist, psychiatrist, psychotherapist, social worker, a medical doctor, or the like. Psychological data may include data gathered from user interactions with persons, documents, and/or computing devices 104; for instance, user patterns of purchases, including electronic purchases, communication such as via chat-rooms or the like, any textual, image, video, and/or data produced by the subject, any textual image, video and/or other data depicting and/or describing the subject, or the like. Any psychological data and/or data used to generate psychological data may be analyzed using machine-learning and/or language processing module as described in this disclosure. As a non-limiting example, biological extraction 116 may include a psychological profile; the psychological profile may be obtained utilizing a questionnaire performed by the user.

Still referring to FIG. 1, in an embodiment, physiological state data may include genomic and/or genetic data, including deoxyribonucleic acid (DNA) samples and/or sequences, such as without limitation DNA sequences or other genetic sequences contained in one or more chromosomes in human cells. As defined in this specification, “genetic data” is defined as data from the user relating to the inherited or acquired genes of a person. “Genomic data,” as used in this disclosure is defined as the study of all the genes from a user including interaction of those genes with each other and with the environment of the user. Genomic and/or genetic data may include, without limitation, ribonucleic acid (RNA) samples and/or sequences, such as samples and/or sequences of messenger RNA (mRNA) or the like taken from human cells. Genetic data may include telomere lengths. Genomic and/or genetic data may include epigenetic data including data describing one or more states of methylation of genetic material. Physiological state data may include proteomic data, which, as used herein, is data describing all proteins produced and/or modified by an organism, colony of organisms, or system of organisms, and/or a subset thereof.

Still referring to FIG. 1, in some embodiments, physiological state data may include microbiome data concerning a microbiome of a person. As used herein, “microbiome data” is any data describing any microorganism and/or combination of microorganisms living on or within a person. Microbiome data may include without limitation biomarkers, genomic data, proteomic data, and/or any other metabolic or biochemical data useful for analysis of the effect of such microorganisms on other physiological state data of a person, as described in further detail below. In some embodiments, computing device 104 may retrieve physiological state data (microbiome data) by analyzing food intake and body measurements of a user. For the purposes of this disclosure, a “food intake” is a consumption of food and beverages. For the purposes of this disclosure, “body measurement” is a measurement of external or internal body structures and compositions. In a non-limiting example, user may manually input food intake and body measurements. In some embodiments, body measurement may include various physical dimensions and characteristics of the human body. In some embodiments, body measurement may include internal measurement; internal dimensions, volumes, and functions of the human body. As a non-limiting example, internal measurement may include bone density, organ size and volume, lung function and capacity, blood glucose level, and the like. In some embodiments, computing device 104 may retrieve internal measurement from a survey, application programming interface (API), and the like.

Still referring to FIG. 1, as used in the current disclosure, an “application programming interface” is a software interface for two or more computer programs to communicate with each other. As a non-limiting example, API may include electronic health record (EHR) APIs, telemedicine APIs, and the like. An application programming interface may be a type of software interface, offering a service to other pieces of software. In contrast to a user interface, which connects a computer to a person, an application programming interface may connect computers or pieces of software to each other. An API may not be intended to be used directly by a person (e.g., the end user) other than a computer programmer who is incorporating it into the software. An API may be made up of different parts which act as tools or services that are available to the programmer. A program or a programmer that uses one of these parts is said to call that portion of the API. The calls that make up the API are also known as subroutines, methods, requests, or endpoints. An API specification may define these calls, meaning that it explains how to use or implement them. One purpose of API may be to hide the internal details of how a system works, exposing only those parts a programmer will find useful and keeping them consistent even if the internal details later change. An API may be custom-built for a particular pair of systems, or it may be a shared standard allowing interoperability among many systems. The term API may be often used to refer to web APIs, which allow communication between computers that are joined by the internet. API may be configured to query for web applications in order to retrieve physiological state data to another web application, database, medical center patient portal, and the like. An API may be further configured to filter through web applications according to a filter criterion. In this disclosure, “filter criteria” are conditions the web applications must fulfill in order to qualify for API. Web applications may be filtered based on these filter criteria. Filter criteria may include, without limitation, types of medical facilities, location of the medical facility, and the like.

With continuing reference to FIG. 1, physiological state data may include one or more user-entered descriptions of a person's physiological state. One or more user-entered descriptions may include, without limitation, user descriptions of symptoms, which may include without limitation current or past physical, psychological, perceptual, and/or neurological symptoms, user descriptions of current or past physical, emotional, and/or psychological problems and/or concerns, user descriptions of past or current treatments, including therapies, nutritional regimens, exercise regimens, pharmaceuticals, food intake, body measurements, or the like, or any other user-entered data that a user may provide to a medical professional when seeking treatment and/or evaluation, and/or in response to medical intake papers, questionnaires, questions from medical professionals, or the like. Physiological state data may include any physiological state data, as described above, describing any multicellular organism living in or on a person including any parasitic and/or symbiotic organisms living in or on the persons; non-limiting examples may include mites, nematodes, flatworms, or the like. Examples of physiological state data described in this disclosure are presented for illustrative purposes only and are not meant to be exhaustive.

Still referring to FIG. 1, physiological state data may include, without limitation, any mental state data. As used in this disclosure, “mental state data” is data describing a state of mind from the user. A mental state of mind may include information about how the user currently feels at the time of the directional inquiry. Factors such as the user's home life, present environment, or the like may affect the state of mind of the user. In addition, the state of mind of the user may be affected by external factors which may include, but are not limited to medications the user is taking, world events that may impact how the user feels mentally, and events in the user's life that may have an impact in the user's mental health such as the death of a family member. Mental state data may include, without limitation, any psychiatric medical data available from the user. Psychiatric data may be used to, for example, avoid a direction inquiry request that may trigger a known/diagnosed mental anomaly. Mental state data may include self-reported paradigmatic states of mind such as, but not limited to love, hate, pleasure, and pain. A mental state may refer to a particular interest or career, for example, the user may feel hated towards the military which may be a factor considered with the directional inquiry request where the computing device may use the mental state to either suggest or eliminate a directional inquiry request response. Mental state data may be self-reported by the user. For example, a user may state how they feel at the time they seek information about a direction. The user may also enter a diagnosis about a mental condition that have obtained from a health provider such as a physician or a psychologist.

Still referring to FIG. 1, computing device 104 may be configured generate using tendency training data correlating mental state data with tendency data and a second machine-learning process, a tendency model. As used in this disclosure, a “tendency model” is defined as how a user behavior may be influenced by certain factors. Factors may include, but not limited to psychological, mental states, social, contextual factors which may include, emotions, habits, routines, and the like. As defined in this disclosure, “tendency data” is data produced as a result of actions by the user, which may include, without limitation, commercial behavior, visits to websites, types of music listened by the user, social media interactions, types of mobile applications installed, and the like. Tendency data may be collected using, for example, surveys, questionnaires, marketing studies, and the like. The machine-learning process and the use of training data will be described further in this disclosure.

Alternative or additionally, and still referring to FIG. 1, computing device 104 may be configured to generate a priority value as a function of the directional response and the tendency model. As used in this disclosure, a “priority value” is a value of a direction based on a directional response and a tendency model for a user. As a non-limiting example, a tendency model for a user may be generated where the user is likely to do well in a job or a career that surrounds them with children. As such, careers such as, for example, a pediatrician or a schoolteacher may receive a higher priority score than a career where the user is not surrounded by people or not surrounded by children. A priority value may include a numerical score on a scale from 1 to 10, where, as in our example, a schoolteacher may receive a score of 10, while a career such as a college professor may receive a lower score. The user may select a filter to remove any priority values that are below a threshold selected by the user. In an embodiment, the computing device is further configured to sort the priority values in descending order. For example, a user may sort the data and view the scores from the highest reported value to the lowest.

Still referring to FIG. 1, physiological state data may include, without limitation any result of any medical test, physiological assessment, cognitive assessment, psychological assessment, or the like. System 100 may receive at least a physiological state data from one or more other devices after performance; system 100 may alternatively or additionally perform one or more assessments and/or tests to obtain at least a physiological state data, and/or one or more portions thereof, on system 100. For instance, at least physiological state data may include or more entries by a user in a form or similar graphical user interface object; one or more entries may include, without limitation, user responses to questions on a psychological, tendency, personality, or cognitive test. For instance, at least a server may present to user a set of assessment questions designed or intended to evaluate a current state of mind of the user, a current psychological state of the user, a personality trait of the user, or the like; at least a server may provide user-entered responses to such questions directly as at least a physiological state data and/or may perform one or more calculations or other algorithms to derive a score or other result of an assessment as specified by one or more testing protocols, such as automated calculation of a Stanford-Binet and/or Wechsler scale for IQ testing, a personality test scoring such as a Myers-Briggs test protocol, or other assessments that may occur to persons skilled in the art upon reviewing the entirety of this disclosure.

Still referring to FIG. 1, retrieval of biological extraction 108 may include, without limitation, reception of biological extraction 108 from another computing device 104 such as a device operated by a medical and/or diagnostic professional and/or entity, a device operated by the user, and/or any device suitable for use as a third-party device as described in further detail below. Biological extraction 108 may be received via a questionnaire posted and/or displayed on a third-party device as described below, inputs to which may be processed as described in further detail below. Alternatively or additionally, biological extraction 108 may be stored in and/or retrieved from a user database 120. User database 120 may include any data structure for ordered storage and retrieval of data, which may be implemented as a hardware or software module. A user database 120 may be implemented, without limitation, as a relational database, a key-value retrieval datastore such as a NOSQL database, or any other format or structure for use as a datastore that a person skilled in the art would recognize as suitable upon review of the entirety of this disclosure. A user database 120 may include a plurality of data entries and/or records corresponding to user tests as described above. Data entries in a user database 120 may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in a user database 120 may reflect categories, cohorts, and/or populations of data consistently with this disclosure. User database 120 may be located in memory of computing device 104 and/or on another device in and/or in communication with system 100.

Still referring to FIG. 1, in some embodiments, computing device 104 may be configured to receive edible data. “Edible data” as used in this disclosure is information relating to consumable items. As a non-limiting example, edible data may include organic information, price, and the like. In some embodiments, edible data may include a meal identification. A “meal identification,” also referred to as meal ID, as used in this disclosure, is a classification of a recipe. In some embodiments, computing device 104 may receive edible data and/or a meal ID from a user. In some embodiments, a meal ID of edible data may include a title and/or a description related to a meal. For example a meal ID may include a name of a dish, such as “Beef Stroganoff.” A description of a meal ID may include one or more general contents of a dish and/or a specific description of the dish. For example, a description may include data that Beef Stroganoff is an originally Russian dish of sautéed pieces of beef served in a sauce of mustard and smetana (sour cream).

Still referring to FIG. 1, computing device may be is configured to receive recipe data containing nutrient data from the user. Recipe data may include a list of nutrients to prepare a meal. For example, and without limitation, edible data may include nutrients in a beef stroganoff dish, which may include: 1 pound uncooked wide egg noodles, ¼ cup butter, divided, 2½ pounds thinly-sliced steak, fine sea salt and freshly-cracked black pepper, 4.5 small white onions, thinly sliced, 3 pound sliced mushrooms, 2 cloves garlic, minced or pressed, ½ cup dry white wine, 1½ cups beef stock, 1 tablespoon Worcestershire sauce, 3 tablespoons all-purpose flour, ½ cup of sour cream, and chopped fresh parsley.

Still referring to FIG. 1, in some embodiments, computing device 104 may extract plurality of nutrients from edible data . . . “Nutrients,” as used in this disclosure, are elements of a meal. Nutrients may include, but are not limited to, meats, vegetables, sauces, syrups, seafoods, fruits, dairy products, and the like. In some embodiments, computing device 104 may utilize a language processing module to extract a plurality of nutrients from edible data.

Still referring to FIG. 1, in some embodiments, edible data may include nutrient data. “Nutrient data,” as used in this disclosure, is information pertaining to the nutritional value of one or more nutrients. In some embodiments, nutrient data may include nutritional values related to nutrients in a meal. For example, nutritional values may include the value of vitamin, caloric, protein, fat, cholesterol, sugar, carbohydrate, sodium, and the like in the meal. For example, in beef stroganoff, the nutritional values may be calories 235, total fat 11 g, saturated fat 6 g, cholesterol 50 mg, sodium 1,044 mg, potassium 336 mg, total carbohydrate 22 g, dietary fiber 1.4 g, sugar 4 g, protein 12 g, vitamin c. In some embodiments, nutritional values may include a daily value of nutrients in a dish. “Daily value (DV),” as used in this disclosure, is the recommended amount of nutrients a person should consume and not to exceed each day. The % DV may be how much a nutrient in a single serving of an individual dish or dietary supplement contributes to a daily diet. For example, if the DV for a certain nutrient is 300 micrograms (mcg) and a dish or supplement has 30 mcg in one serving, the % DV for that nutrient in a serving of the product may be 10%. In some embodiments, computing device 104 may receive recipe data from a user database. User database may contain recipe data received from a plurality of different users categorized to a common meal ID. For example, user database may contain a recipe data table containing a plurality of different recipes and nutritional values common for a beef stroganoff dish.

Still referring to FIG. 1, in some embodiments, computing device 104 may be configured to classify plurality of nutrients to impact factors. An “impact factor,” as used in this disclosure, is a metric of influence one or more nutrients has on an individual's biological system. A “biological system” as used in this disclosure is a process and/or group of processes that occur in an individual's physiology. Impact factors may include, without limitation, concentration of nutrients, quantity of nutrients, calories of nutrients, allergens associated with one or more nutrients, carbohydrate and/or other macronutrient quantities, ratios, and the like. In some embodiments, impact factors may be based on essential macronutrients and micronutrients. “Micronutrients,” as used herein, are nutrients that a person needs in small doses. For example, micronutrients may include vitamins and minerals. “Macronutrients,” as used herein, are nutrients that a person needs in larger amounts. For example, macronutrients may include water, protein, carbohydrates, and fats. In some embodiments, impact factors may be based on nutrients essential for boosting the immune system, helping prevent or delay certain cancers, such as prostate cancer, strengthening teeth and bones, aiding in calcium absorption, maintaining healthy skin, helping the body metabolize proteins and carbs, supporting healthy blood, burning fat, building muscle, maintaining weight, losing water weight, aiding brain and nervous system functioning, aiding in blood clotting, helping to carry oxygen and/or the like. A user may select, through GUI, what impact factors may be based on. In some embodiments, a user may select a plurality of impact factors. In some embodiments, receive impart factor data in the form of documents, medical papers, research papers, and the like through an impact factors database. “Impact factor database,” as used in this disclosure, is a data structure containing information related to a plurality of impact factors. An impact factor database may be populated by computing device 104 utilizing a web crawler. An impact factor database may be populated by expert submission. An “expert,” as used herein, is a person who has a comprehensive and authoritative knowledge of or skill in a particular area. For example, a doctor may submit a paper on how fish oil aids in preventing cancer. An expert submission may include a single expert submission and/or a plurality of submissions from an expert; plurality of submissions may be received from a plurality of experts as described in U.S. patent application Ser. No. 16/397,814, filed, Apr. 29, 2019, and titled “METHODS AND SYSTEMS FOR CLASSIFICATION USING EXPERT DATA”, of which is incorporated by reference herein in its entirety.

Still referring to FIG. 1, in some embodiments, computing device 104 may classify plurality of nutrients to impact factors utilizing a nutrient classifier. A “classifier,” as used in this disclosure is a machine-learning model, such as a mathematical model, neural net, or program generated by a machine learning algorithm known as a “classification algorithm,” as described in further detail below, that sorts inputs into categories or bins of data, outputting the categories or bins of data and/or labels associated therewith. A nutrient classifier may be configured to output at least a datum that labels or otherwise identifies a set of data that are clustered together, found to be close under a distance metric as described below, or the like. In some embodiments, a nutrient classifier may receive meal ID and recipe data as an input and output a plurality of matched nutrient data elements to an importance factor. For example, a nutrient classifier may match nutrients in a dish that contain a nutritional value that are essential for boosting the immune system. In some embodiments, a nutrient classifier may receive meal ID and recipe data as an input and output a plurality of matched nutrient data elements to a plurality of impact factors. For example, a nutrient classifier may match nutrients in a dish that contain a nutritional value that are essential for boosting the immune system, building muscle, maintaining healthy skin, and the like. Training data for a nutrient classifier may include data from impact factor data. For example, classification based on muscle building may include training data containing documents and expert submission exemplifying nutrients that may correlate to muscle building. In some embodiments, training data may include, a plurality of recipe data received from a plurality of user from user database. Additional disclosure related to edible data and recipe data can be found in U.S. patent application Ser. No. 18/090,411, filed on Dec. 28, 2022, and titled “APPARATUS AND METHOD FOR SCORING A NUTRIENT,” having an attorney docket number of 1057-213USU1, of which is incorporated by reference herein in its entirety.

Still referring to FIG. 1, in some embodiments, computing device 104 may be configured to generate a stress level datum 124 related to a user. For the purposes of this disclosure, a “stress level datum” is a data element that is related to quantifiable indicators that measure a user's level of stress. In some embodiments, stress level datum 124 may be related to a user's vocation. In some embodiments, stress level datum 124 may numerical value or characteristic value. As a non-limiting example, stress level datum 124 may be a ‘2’ for a score range of 0-10, where ‘0’ may represent a user having a minimum and/or no stress and ‘10’ represents user having a lot of stress. In other non-limiting embodiments, stress level datum 124 may be a quality characteristic, such as a color coding, where each color is associated with a level of stress. As a non-limiting example, stress level datum 124 may be green, where green may represent a minimum and/or no stress. As another non-limiting example, stress level datum 124 may be red, where red may represent a lot of stress. As another non-limiting example, stress level datum 124 may be light grey when there is no stress and the color may get darker as stress level increases. In some embodiments, stress level datum 124 may include low to high scoring. As a non-limiting example, stress level datum 124 may be ‘low’ when there is minimum and/or no stress and stress level datum 124 may be ‘high’ when there is a lot of stress. In some embodiments, stress level datum 124 may be updated in real-time as computing device 104 receives new biological extraction 116. In some embodiments, user may manually input stress level datum 124. In some embodiments, computing device 104 may retrieve stress level datum 124 from user database 120.

Still referring to FIG. 1, in some embodiments, computing device 104 may generate stress level datum 124 by analyzing physiological state data of biological extraction 116 and/or directional inquiry 108. As a non-limiting example, computing device 104 may analyze heart rate variability, cortisol level, blood pressure, respiratory rate, and the like to determine stress level datum 124. As another non-limiting example, computing device 104 may analyze directional inquiry 108 to determine stress level datum 124. In some embodiments, computing device 104 may analyze survey to determine stress level datum 124.

With continued reference to FIG. 1, in some embodiments, computing device 104 may use a language processing module to find a keyword to determine stress level datum 124. The language processing module may be configured to extract one or more words from the one or more documents, survey, physiological state data of biological extraction 116, directional inquiry 108, and the like. One or more words may include, without limitation, strings of one or more characters, including without limitation any sequence or sequences of letters, numbers, punctuation, diacritic marks, engineering symbols, medical symbols and abbreviations, formulas, spaces, whitespace, and other symbols, including any symbols thereof. Textual data may be parsed into tokens, which may include a simple word (sequence of letters separated by whitespace) or more generally a sequence of characters as described previously. The term “token,” as used herein, refers to any smaller, individual groupings of text from a larger source of text; tokens may be broken up by word, pair of words, sentence, or other delimitation. These tokens may in turn be parsed in various ways. Textual data may be parsed into words or sequences of words, which may be considered words as well. Textual data may be parsed into “n-grams,” where all sequences of n consecutive characters are considered. Any or all possible sequences of tokens or words may be stored as “chains,” for example for use as a Markov chain or Hidden Markov Model.

With continued reference to FIG. 1, language processing module may operate to produce a language processing model. Language processing model may include a program automatically generated by computing device 104 and/or language processing module to produce associations between one or more words extracted from at least a document and detect associations, including without limitation mathematical associations, between such words. Associations between language elements, where language elements include for purposes herein extracted words, relationships of such categories to other such term may include, without limitation, mathematical associations, including without limitation statistical correlations between any language element and any other language element and/or language elements. Statistical correlations and/or mathematical associations may include probabilistic formulas or relationships indicating, for instance, a likelihood that a given extracted word indicates a given category of semantic meaning. As a further example, statistical correlations and/or mathematical associations may include probabilistic formulas or relationships indicating a positive and/or negative association between at least an extracted word and/or a given semantic meaning; positive or negative indication may include an indication that a given document is or is not indicating a category semantic meaning. Whether a phrase, sentence, word, or other textual element in a document or corpus of documents constitutes a positive or negative indicator may be determined, in an embodiment, by mathematical associations between detected words, comparisons to phrases and/or words indicating positive and/or negative indicators that are stored in memory at computing device, or the like.

With continued reference to FIG. 1, language processing module may generate the language processing model by any suitable method, including without limitation a natural language processing classification algorithm; language processing model may include a natural language process classification model that enumerates and/or derives statistical relationships between input terms and output terms. Algorithm to generate language processing model may include a stochastic gradient descent algorithm, which may include a method that iteratively optimizes an objective function, such as an objective function representing a statistical estimation of relationships between terms, including relationships between input terms and output terms, in the form of a sum of relationships to be estimated. In an alternative or additional approach, sequential tokens may be modeled as chains, serving as the observations in a Hidden Markov Model (HMM). HMMs, as used herein, are statistical models with inference algorithms that that may be applied to the models. In such models, a hidden state to be estimated may include an association between an extracted words, phrases, and/or other semantic units. There may be a finite number of categories to which an extracted word may pertain; an HMM inference algorithm, such as the forward-backward algorithm or the Viterbi algorithm, may be used to estimate the most likely discrete state given a word or sequence of words. Language processing module may combine two or more approaches. For instance, and without limitation, machine-learning program may use a combination of Naive-Bayes (NB), Stochastic Gradient Descent (SGD), and parameter grid-searching classification techniques; the result may include a classification algorithm that returns ranked associations.

With continued reference to FIG. 1, generating language processing model may include generating a vector space, which may be a collection of vectors, defined as a set of mathematical objects that can be added together under an operation of addition following properties of associativity, commutativity, existence of an identity element, and existence of an inverse element for each vector, and can be multiplied by scalar values under an operation of scalar multiplication compatible with field multiplication, and that has an identity element is distributive with respect to vector addition, and is distributive with respect to field addition. Each vector in an n-dimensional vector space may be represented by an n-tuple of numerical values. Each unique extracted word and/or language element as described above may be represented by a vector of the vector space. In an embodiment, each unique extracted and/or other language element may be represented by a dimension of vector space; as a non-limiting example, each element of a vector may include a number representing an enumeration of co-occurrences of the word and/or language element represented by the vector with another word and/or language element. Vectors may be normalized, scaled according to relative frequencies of appearance and/or file sizes. In an embodiment associating language elements to one another as described above may include computing a degree of vector similarity between a vector representing each language element and a vector representing another language element; vector similarity may be measured according to any norm for proximity and/or similarity of two vectors, including without limitation cosine similarity, which measures the similarity of two vectors by evaluating the cosine of the angle between the vectors, which can be computed using a dot product of the two vectors divided by the lengths of the two vectors. Degree of similarity may include any other geometric measure of distance between vectors.

With continued reference to FIG. 1, language processing module may use a corpus of documents to generate associations between language elements in a language processing module may then use such associations to analyze words extracted from one or more documents and determine that the one or more documents indicate significance of a category. In an embodiment, language module and/or computing device 104 may perform this analysis using a selected set of significant documents, such as documents identified by one or more experts as representing good information; experts may identify or enter such documents via graphical user interface or may communicate identities of significant documents according to any other suitable method of electronic communication, or by providing such identity to other persons who may enter such identifications into computing device 104. Documents may be entered into a computing device by being uploaded by an expert or other persons using, without limitation, file transfer protocol (FTP) or other suitable methods for transmission and/or upload of documents; alternatively or additionally, where a document is identified by a citation, a uniform resource identifier (URI), uniform resource locator (URL) or other datum permitting unambiguous identification of the document, computing device 104 may automatically obtain the document using such an identifier, for instance by submitting a request to a database or compendium of documents such as JSTOR as provided by Ithaka Harbors, Inc. of New York.

With continued reference to FIG. 1, in some embodiments, computing device 104 may be configured to generate stress level training data. In a non-limiting example, stress level training data may include correlations between exemplary user data, exemplary biological extractions and exemplary stress level datums. In some embodiments, stress level training data may be stored in database. In some embodiments, stress level training data may be received from one or more users, database, external computing devices, and/or previous iterations of processing. As a non-limiting example, stress level training data may include instructions from a user, who may be an expert user, a past user in embodiments disclosed herein, or the like, which may be stored in memory and/or stored in database, where the instructions may include labeling of training examples. In some embodiments, stress level training data may be updated iteratively using a feedback loop. As a non-limiting example, computing device 104 may update stress level training data iteratively through a feedback loop as a function of user data 106, directional inquiry 108, biological extraction 116, or the like. In some embodiments, computing device 104 may be configured to generate stress level machine-learning model. In a non-limiting example, generating stress level machine-learning model may include training, retraining, or fine-tuning stress level machine-learning model using stress level training data or updated stress level training data. In some embodiments, computing device 104 may be configured to determine stress level datum 124 using stress level machine-learning model (i.e. trained or updated stress level machine-learning model). In some embodiments, generating training data and training machine-learning models may be simultaneous.

Still referring to FIG. 1, in some embodiments, computing device 104 is configured to generate a nutrient program 128 as a function of vocation data 118 of user data 106. For the purposes of this disclosure, a “nutrient program” is an outline or schedule of what a user will cat for meals or snacks over a specific period. In some embodiments, computing device 104 may be configured to generate a nutrient program 128 as a function of health history information, pharmaceutical drug history, and/or family health history. In some embodiments, computing device 104 may be configured to generate nutrient program 128 as a function of stress level datum 124. In some embodiments, computing device 104 may generate nutrient program 128 as a function of user data 106, stress level datum 124, and the like; for instance, time available to cook, experience cooking, average number of meals consumed in a day, weekday habits, weekend habits, snacking habits, allergens, intolerances, pharmaceuticals, disease states, stress levels, emotional states, sleep habits genetics, and the like. In some embodiments, computing device 104 may generate a nutrient program 128 as a function of a biological demand from physiological state data. For the purposes of this disclosure, a “biological demand” is biological requirements or needs of a user. As a non-limiting example, biological demand may include nutrient demand; such as micronutrients, macronutrients, and the like. In some embodiments, computing device 104 may determine biological demand of user or physiological state data through the use of machine-learning module. In some embodiments, user may manually input biological demand. In some embodiments, biological demand may be retrieved from database. In some embodiments, computing device 104 may determine nutrient program 128 as a function of edible data or impact factor. In some embodiments, computing device 104 may determine a food or recipe that user may most likely will enjoy as a function of user data 106 or stress level datum 124 and generate nutrient program 128 as a function of the food or recipe. In some embodiments, nutrient program 128 may help maintaining user's stress level datum 124 to be ‘low.’ In some embodiments, nutrient program 128 may help bringing stress level datum 124 from ‘high’ to ‘medium’ or ‘low.’ In some embodiments, nutrient program 128 may eliminate toxins from a user's body. In some embodiments, computing device 104 may generate nutrient program 128 depends on what a user's job is. As a non-limiting example, a user's job may need more brain power, then computing device 104 may generate nutrient program 128 that includes more salmon, eggs, and the like into diet. In some embodiments, nutrient program 128 may consider calories, macronutrients, micronutrients, vitamins, minerals, protein, fat, cholesterol, sugar, carbohydrate, sodium, and the like. As a non-limiting example, nutrient program 128 may include a list of food, meal structure, recipe, portion sizes, nutrient distributions, and the like. In some embodiments, user may manually input nutrient program 128. In some embodiments, nutrient program 128 may be retrieved from database.

Still referring to FIG. 1, additional disclosure related to nutrient program 128 may be found in U.S. patent application Ser. No. 18/661,324, filed on May 10, 2024, and titled “METHODS AND SYSTEMS FOR DETERMINING A COMPATIBLE SUBSTANCE,” having an attorney docket number of 1057-053USC1, and U.S. patent application Ser. No. 17/517,801, filed on Nov. 3, 2024, and titled “METHOD OF SYSTEM FOR REVERSING INFLAMMATION IN A USER,” having an attorney docket number of 1057-138USC1, the entirety of each of which is incorporated by reference herein in its entirety.

Still referring to FIG. 1, in some embodiments, computing device 104 may generate nutrient program 128 using a machine-learning module. In some embodiments, computing device 104 may be configured to generate program training data. In a non-limiting example, program training data may include correlations between exemplary user data, exemplary vocation data, exemplary stress level datums, exemplary biological demands, exemplary edible data, exemplary impact factors and/or exemplary nutrient programs. In some embodiments, program training data may be stored in database. In some embodiments, program training data may be received from one or more users, database, external computing devices, and/or previous iterations of processing. As a non-limiting example, program training data may include instructions from a user, who may be an expert user, a past user in embodiments disclosed herein, or the like, which may be stored in memory and/or stored in database, where the instructions may include labeling of training examples. In some embodiments, program training data may be updated iteratively using a feedback loop. As a non-limiting example, computing device 104 may update program training data iteratively through a feedback loop as a function of physiological state data, user data 106, directional inquiry 108, biological extraction 116, stress level datum 124, user cohort, and the like. In some embodiments, computing device 104 may be configured to generate program machine-learning model. In a non-limiting example, generating program machine-learning model may include training, retraining, or fine-tuning program machine-learning model using program training data or updated program training data. In some embodiments, computing device 104 may be configured to determine nutrient program 128 using program machine-learning model (i.e. trained or updated program machine-learning model).

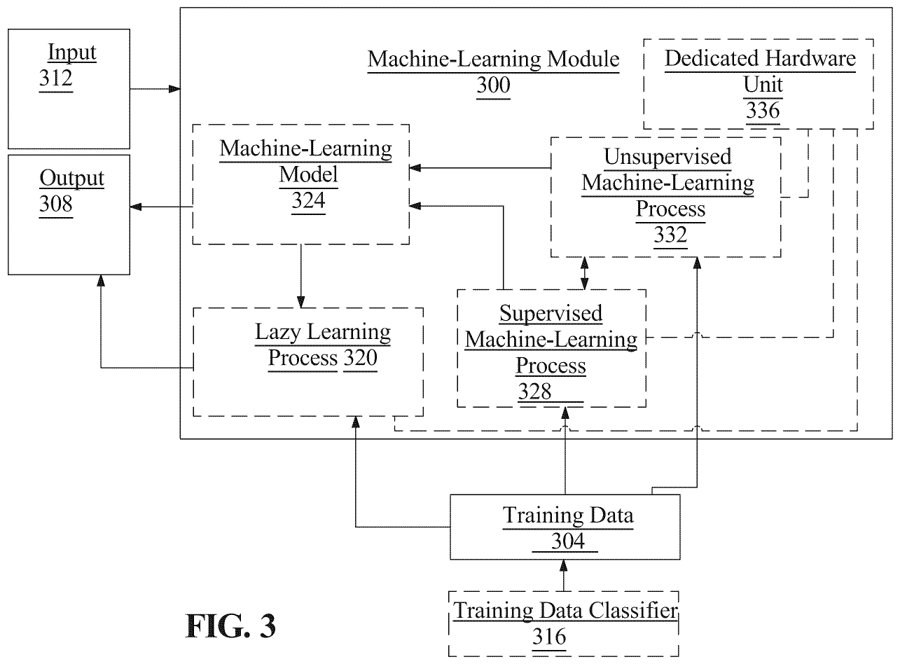

Still referring to FIG. 1, in some embodiments, user, user data 106 or biological extraction 116 may be classified to a user cohort using a cohort classifier. Cohort classifier may be consistent with any classifier discussed in this disclosure. Cohort classifier may be trained on cohort training data, wherein the cohort training data may include user data 106 or biological extraction 116 correlated to user cohorts. In some embodiments, a user, user data 106 or biological extraction 116 may be classified to a user cohort and computing device 104 may determine nutrient program 128 based on the user cohort using a machine-learning module as described in detail with respect to FIG. 3 and the resulting output may be used to update program training data.

Still referring to FIG. 1, in some embodiments, computing device 104 may generate an outcome datum of nutrient program 128. For the purposes of this disclosure, an “outcome datum” is a degree to which a nutrient program can affect various aspects of a user's biological extraction. As a non-limiting example, outcome datum may indicate the predicted change of stress level datum 124 of user by implementing nutrient program 128. As another non-limiting example, outcome datum may include a predicted percentage value that indicates the improvement of user's health or mental health by implementing nutrient program 128. As another non-limiting example, nutrient program 128 may include an impact of nutrient program 128 for toxin elimination. In some embodiments, user may manually input outcome datum. In some embodiments, outcome datum may be retrieved from database.

Still referring to FIG. 1, in some embodiments, computing device 104 may generate outcome datum using a machine-learning module. In some embodiments, computing device 104 may be configured to generate outcome training data. In a non-limiting example, outcome training data may include correlations between exemplary user data, exemplary biological extractions, exemplary stress level datum, exemplary nutrient program and/or exemplary outcome datums. In some embodiments, outcome training data may be stored in database. In some embodiments, outcome training data may be received from one or more users, database, external computing devices, and/or previous iterations of processing. As a non-limiting example, outcome training data may include instructions from a user, who may be an expert user, a past user in embodiments disclosed herein, or the like, which may be stored in memory and/or stored in database, where the instructions may include labeling of training examples. In some embodiments, outcome training data may be updated iteratively using a feedback loop. As a non-limiting example, computing device 104 may update outcome training data iteratively through a feedback loop as a function of physiological state data, stress level datum 124, user cohort, and the like. In some embodiments, computing device 104 may be configured to generate outcome machine-learning model. In a non-limiting example, generating outcome machine-learning model may include training, retraining, or fine-tuning outcome machine-learning model using outcome training data or updated outcome training data. In some embodiments, computing device 104 may be configured to determine outcome datum using outcome machine-learning model (i.e. trained or updated outcome machine-learning model).

Still referring to FIG. 1, in some embodiments, computing device 104 may be configured to analyze biological extraction 116 or user data 106 to determine a strength datum. For the purposes of this disclosure, a “strength datum” is a data element related to a user's mental or physical strength. As a non-limiting example, strength datum may include mental stamina, physical health or physical stamina of a user. In some embodiments, computing device 104 may determine strength datum through the use of machine-learning process. In some embodiments, user or third-party may manually input strength datum. In some embodiments, computing device 104 may retrieve strength datum from database. In some embodiments, computing device 104 may be configured to generate a vocation guide as a function of strength datum. For the purposes of this disclosure, a “vocation guide” is a suggestion for a user related to one's vocation. As a non-limiting example, vocation guide may include a suggestion not to take a new job or new responsibility for a user's mental or physical health. As another non-limiting example, vocation guide may include a suggestion not to take a new job or new responsibility based on analysis of a user's mental stamina or physical stamina. In some embodiments, computing device 104 may generate vocation guide using stress level datum 124. In some embodiments, user or third-party may manually input vocation guide. In some embodiments, computing device 104 may retrieve vocation guide from database. In some embodiments, computing device 104 may determine vocation guide through the use of machine-learning process.

Still referring to FIG. 1, in some embodiments, computing device 104 may pair a third-party with a user as a function of stress level datum 124 and vocation data 118. For the purposes of this disclosure, a “third-party” is any individual, entity or organization that has expertise in managing physical health or mental health. In a non-limiting example, computing device 104 may pair a third-party with a user if stress level datum 124 includes ‘high,’ indicating that intervention by third-party is recommended to manage stress or other negative effects. In another non-limiting example, computing device 104 may pair a third-party with a user based on a vocation of the user. For example, and without limitation, computing device 104 may pair a third-party that has an expertise in a specific vocation to a user that has the vocation. In some embodiments, pairing a third-party with a user includes identifying a medial professional with experience providing guidance as to stress level datum 124, vocation data 118 or directional inquiry 108. In some embodiments, pairing a third-party with a user may include collecting identification information of a user (e.g., user data 106), and transmitting the identification information of the user to the third-party. In some embodiments, pairing a third-party with a user may include collecting identification information of a third-party, and transmitting the identification information of the medial professional to the user. Identification information may include, in non-limiting examples, a name, phone number, email address, account number (such as an account number on a messaging platform), and a social media account handle. In some embodiments, pairing a third-party with a user may include opening a communication channel between the medial professional and the user, such as by starting a phone call between the two. In some embodiments, pairing a third-party with a user may include scheduling a meeting including the medial professional and the user. In some embodiments, a third-party may be paired with more than one user. For example, in some embodiments, two or more users may have similar normality level datums 1008, and a medial professional may be paired with more than one such user. A third-party may include an artificial intelligence system including any simulation of human intelligence and/or problem-solving capabilities processed by a machine, such as a computer system.

Still referring to FIG. 1, computing device 104 is configured to output directional response 132. In some embodiments, computing device 104 may be configured to output nutrient program 128, stress level datum 124, information of paired third-party, and the like to a user device 112. In some embodiments, computing device 104 may generate a notification datum and transmit the notification datum to a user device 112. For the purposes of this disclosure, a “notification datum” is a data element that informs a user the generation of data for the user. As a non-limiting example, notification datum may include sound, banner, message, call, text, vibration, and the like. In some embodiments, computing device 104 may be further configured to generate a user interface displaying nutrient program 128, stress level datum 124, information of paired third-party, and the like. For the purposes of this disclosure, a “user interface” is a means by which a user and a computer system interact; for example through the use of input devices and software. A user interface may include a graphical user interface (GUI), command line interface (CLI), menu-driven user interface, touch user interface, voice user interface (VUI), form-based user interface, any combination thereof and the like. In some embodiments, user interface may operate on and/or be communicatively connected to a decentralized platform, metaverse, and/or a decentralized exchange platform associated with the user. For example, a user may interact with user interface in virtual reality. In some embodiments, a user may interact with the use interface using a computing device distinct from and communicatively connected to at least a computing device 104. For example, a smart phone, smart, tablet, or laptop operated by a user. In an embodiment, user interface may include a graphical user interface. A “graphical user interface,” as used herein, is a graphical form of user interface that allows users to interact with electronic devices. In some embodiments, GUI may include icons, menus, other visual indicators or representations (graphics), audio indicators such as primary notation, and display information and related user controls. A menu may contain a list of choices and may allow users to select one from them. A menu bar may be displayed horizontally across the screen such as pull-down menu. When any option is clicked in this menu, then the pull-down menu may appear. A menu may include a context menu that appears only when the user performs a specific action. An example of this is pressing the right mouse button. When this is done, a menu may appear under the cursor. Files, programs, web pages and the like may be represented using a small picture in a graphical user interface. For example, links to decentralized platforms as described in this disclosure may be incorporated using icons. Using an icon may be a fast way to open documents, run programs etc. because clicking on them yields instant access.

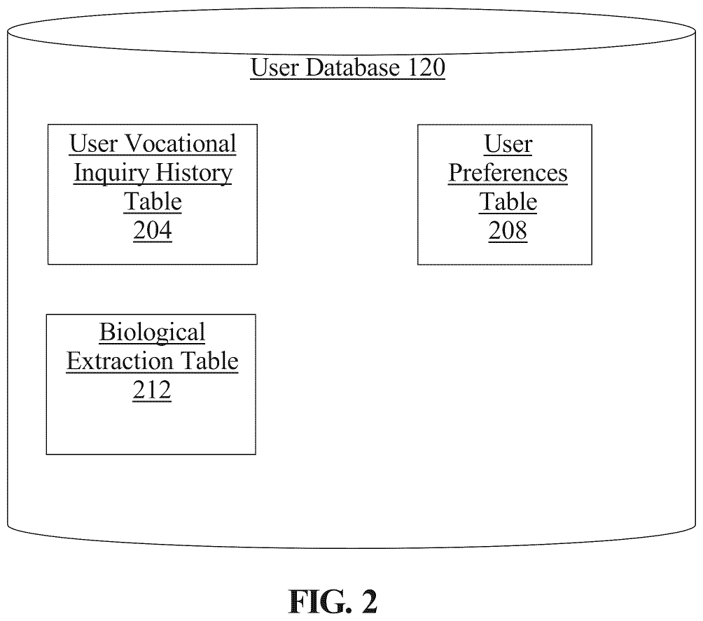

Referring now to FIG. 2, an exemplary embodiment of a user database 120 is illustrated. One or more tables in user database 120 may include, without limitation, a user directional inquiry history table 204, which may be used to store data describing past user requests, educational activities, employment history, prior directional response 132, or the like. One or more tables in user database 120 may include, without limitation, user preference table 208, which may be used to store one or more user preferences regarding type of employment, interests, or the like. One or more tables in user database 120 may include, without limitation, a biological extraction table 212, which may be used to store biological extraction data 116. Persons skilled in the art, upon reviewing the entirety of this disclosure, will be aware of various alternative or additional data which may be stored in user database 120, including without limitation any data concerning any user activity, demographics, profile information, viewing and/or media consumption history, or the like.

Still referring to FIG. 1, computing device 104 is configured to generate a directional response 132 as a function of nutrient program 128. In some embodiments, computing device 104 may be configured to generate directional response 132 as a function of outcome datum. This may be accomplished, without limitation, using machine learning. A machine learning process 136 may be trained using directional training data 140 correlating a plurality of biological extractions, outcome datums, or nutrient programs to a plurality of directions. Computing device may be configured to output the directional response 132 as a function of the biological extraction 116 from the user and the machine learning process 136. A “machine learning process” is a process that automatedly uses a body of data known as “training data” and/or a “training set” to generate an algorithm that will be performed by a computing device 104/module to produce outputs given data provided as inputs; this is in contrast to a non-machine learning software program where the commands to be executed are determined in advance by a user and written in a programming language. This contrasts with a non-machine learning software program where the commands to be executed are determined in advance by a user and written in a programming language. At least a machine learning process 136 may be used by computing device 104 to generate an inquiry response as described in further detail below.

With reference to FIG. 1, directional training data 140 may be used to train a machine learning process 136 where the training data correlates a plurality of biological extraction to a plurality of directions. As a non-limiting illustrative example, the directional response is the output as a function of the biological extraction 116 from the user and the machine learning process 136.

Still referring to FIG. 1, computing device 104 may be configured to update a directional response 132 to generate updated directional response 144 as a function of the preferences of the user. As used in this disclosure, a “preference of the user” are themes that a user may choose to sort the directional responses. Theme may include financial factors such as, but not limited to, income, and potential income potential. As a non-limiting example, preference of the user may include, flavor profile preferences, flavor profile dislikes, ingredient preferences, ingredient dislikes, texture preferences, texture dislikes, cuisine preferences, cuisine dislikes, molecule interaction, dining-out tendencies, take-out tendencies, DIY tendencies, and the like. Another category may include geographical location such as a particular city, the number of miles away from the user's residence, and the like. For example, the user may prefer to update the directional response according to an income level. In another non-limiting example, a user may update the directional response 132 (e.g., updated directional response 144) according to their religious beliefs. A user, for example, may be a Jehovah's Witness, and may want to update the directional response to refine the direction inquiry response to not show any directions performing a blood transfusion. Other non-limiting examples of user preferences may include, updating by the number of hours of work required, a requirement to join a labor union, update to show directions that only work on weekends, and the like. Alternatively, or additionally, a user may update the directional response by selecting multiple categories. For example, a user may select to view potential directions based on income and distance from a geographical location, such as the residence of the user, that includes a certain range. As a non-limiting example, the user may select a direction of $100,000 where the user may travel up to 30 miles from their residence.

Alternatively, or additionally, computing device 104 may generate a directional response 132 where the response includes an educational recommendation. Systems and methods for generating educational inquiries and corresponding responses may be implemented, without limitation, as described in U.S. Nonprovisional application Ser. No. 16/825,098, filed on Mar. 20, 2020, and entitled “ARTIFICIAL INTELLIGENCE SYSTEMS AND METHODS FOR GENERATING EDUCATIONAL INQUIRY RESPONSES FROM BIOLOGICAL EXTRACTIONS,” which is hereby incorporated by reference in its entirety.

With reference back to FIG. 1, in an embodiment, the preferences of the user may include a type of work experience. As used in this disclosure, a “type of work experience” is defined as a type of career or job placement that user seeks a directional response 132. For example, user may seek a work shadowing experience. Work shadowing allows user to observe the work of another user giving user an insight into what working life in that career or job or with a particular employer may be like. Another example of a type of job experience may include working abroad. Working abroad would allow user to travel to a foreign country while engaging in employment. Another non-limiting example of a type of work experience may include volunteer work. Volunteer work would expose user to charity work or work in the public sector without the benefit of financial compensation. Other non-limiting examples of types of work experience include part-time work, full time work, an internship, a fellowship, and the like. In an embodiment, computing device 104 may be configured to update a directional response as a function of the type of work experience

Still referring to FIG. 1, computing device 104 may be configured to output the machine-learning classifier to the device of user 112. The output may include, for example, a response in textual form that may include the directional response. The output may include contact information for a direction professional such as a job counselor, a life coach, a recruiter, and the like. In an embodiment, the output transmitted to the user includes a plurality of hyperlinks as a function of the machine-learning classifier. A hyperlink, as used in this disclosure, is a link from a hypertext file or document to another location or file, typically activated by clicking on a highlighted word or image on the screen. The hyperlinks may include, but not limited to, links to specific websites where more information may be available regarding a particular direction. In another non-limiting example, the hyperlinks may include contact information to agencies that specialize with the direction which is the subject of the response. In another non-limiting example, a hyperlink may contain a link to live help where a professional may appear and answer user questions.

Referring now to FIG. 3, an exemplary embodiment of a machine-learning module 300 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 304 to generate an algorithm instantiated in hardware or software logic, data structures, and/or functions that will be performed by a computing device/module to produce outputs 308 given data provided as inputs 312; this is in contrast to a non-machine learning software program where the commands to be executed are determined in advance by a user and written in a programming language.

Still referring to FIG. 3, “training data,” as used herein, is data containing correlations that a machine-learning process may use to model relationships between two or more categories of data elements. For instance, and without limitation, training data 304 may include a plurality of data entries, also known as “training examples,” each entry representing a set of data elements that were recorded, received, and/or generated together; data elements may be correlated by shared existence in a given data entry, by proximity in a given data entry, or the like. Multiple data entries in training data 304 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 304 according to various correlations; correlations may indicate causative and/or predictive links between categories of data elements, which may be modeled as relationships such as mathematical relationships by machine-learning processes as described in further detail below. Training data 304 may be formatted and/or organized by categories of data elements, for instance by associating data elements with one or more descriptors corresponding to categories of data elements. As a non-limiting example, training data 304 may include data entered in standardized forms by persons or processes, such that entry of a given data element in a given field in a form may be mapped to one or more descriptors of categories. Elements in training data 304 may be linked to descriptors of categories by tags, tokens, or other data elements; for instance, and without limitation, training data 304 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 3, training data 304 may include one or more elements that are not categorized; that is, training data 304 may not be formatted or contain descriptors for some elements of data. Machine-learning algorithms and/or other processes may sort training data 304 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 304 to be made applicable for two or more distinct machine-learning algorithms as described in further detail below. Training data 304 used by machine-learning module 300 may correlate any input data as described in this disclosure to any output data as described in this disclosure. As a non-limiting illustrative example, input data may include biological extraction 116, user data 106, vocation data 118, directional inquiry 108, stress level datum 124, nutrient program 128, and the like. As a non-limiting illustrative example, output data may include stress level datum 124, nutrient program 128, outcome datum, guided recommendation, and the like.

Further referring to FIG. 3, training data may be filtered, sorted, and/or selected using one or more supervised and/or unsupervised machine-learning processes and/or models as described in further detail below; such models may include without limitation a training data classifier 316. Training data classifier 316 may include a “classifier,” which as used in this disclosure is a machine-learning model as defined below, such as a data structure representing and/or using a mathematical model, neural net, or program generated by a machine learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. A distance metric may include any norm, such as, without limitation, a Pythagorean norm. Machine-learning module 300 may generate a classifier using a classification algorithm, defined as a processes whereby a computing device and/or any module and/or component operating thereon derives a classifier from training data 304. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 316 may classify elements of training data to user cohorts. For example, and without limitation, user cohorts may be related to a user's age, gender, weight, medical history, existing conditions, and the like.

Still referring to FIG. 3, Computing device may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A) P(A)÷P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device may then calculate a likelihood table by calculating probabilities of different data entries and classification labels. Computing device may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 3, Computing device may be configured to generate a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 3, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute/as derived using a Pythagorean norm: l=\sqrt{Σᵢ₌₀ⁿaᵢ²}, where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

With further reference to FIG. 3, training examples for use as training data may be selected from a population of potential examples according to cohorts relevant to an analytical problem to be solved, a classification task, or the like. Alternatively or additionally, training data may be selected to span a set of likely circumstances or inputs for a machine-learning model and/or process to encounter when deployed. For instance, and without limitation, for each category of input data to a machine-learning process or model that may exist in a range of values in a population of phenomena such as images, user data, process data, physical data, or the like, a computing device, processor, and/or machine-learning model may select training examples representing each possible value on such a range and/or a representative sample of values on such a range. Selection of a representative sample may include selection of training examples in proportions matching a statistically determined and/or predicted distribution of such values according to relative frequency, such that, for instance, values encountered more frequently in a population of data so analyzed are represented by more training examples than values that are encountered less frequently. Alternatively or additionally, a set of training examples may be compared to a collection of representative values in a database and/or presented to a user, so that a process can detect, automatically or via user input, one or more values that are not included in the set of training examples. Computing device, processor, and/or module may automatically generate a missing training example; this may be done by receiving and/or retrieving a missing input and/or output value and correlating the missing input and/or output value with a corresponding output and/or input value collocated in a data record with the retrieved value, provided by a user and/or other device, or the like.

Continuing to refer to FIG. 3, computer, processor, and/or module may be configured to preprocess training data. “Preprocessing” training data, as used in this disclosure, is transforming training data from raw form to a format that can be used for training a machine learning model. Preprocessing may include sanitizing, feature selection, feature scaling, data augmentation and the like.

Still referring to FIG. 3, computer, processor, and/or module may be configured to sanitize training data. “Sanitizing” training data, as used in this disclosure, is a process whereby training examples are removed that interfere with convergence of a machine-learning model and/or process to a useful result. For instance, and without limitation, a training example may include an input and/or output value that is an outlier from typically encountered values, such that a machine-learning algorithm using the training example will be adapted to an unlikely amount as an input and/or output; a value that is more than a threshold number of standard deviations away from an average, mean, or expected value, for instance, may be eliminated. Alternatively or additionally, one or more training examples may be identified as having poor quality data, where “poor quality” is defined as having a signal to noise ratio below a threshold value. Sanitizing may include steps such as removing duplicative or otherwise redundant data, interpolating missing data, correcting data errors, standardizing data, identifying outliers, and the like. In a nonlimiting example, sanitization may include utilizing algorithms for identifying duplicate entries or spell-check algorithms.

As a non-limiting example, and with further reference to FIG. 3, images used to train an image classifier or other machine-learning model and/or process that takes images as inputs or generates images as outputs may be rejected if image quality is below a threshold value. For instance, and without limitation, computing device, processor, and/or module may perform blur detection, and eliminate one or more Blur detection may be performed, as a non-limiting example, by taking Fourier transform, or an approximation such as a Fast Fourier Transform (FFT) of the image and analyzing a distribution of low and high frequencies in the resulting frequency-domain depiction of the image; numbers of high-frequency values below a threshold level may indicate blurriness. As a further non-limiting example, detection of blurriness may be performed by convolving an image, a channel of an image, or the like with a Laplacian kernel; this may generate a numerical score reflecting a number of rapid changes in intensity shown in the image, such that a high score indicates clarity and a low score indicates blurriness. Blurriness detection may be performed using a gradient-based operator, which measures operators based on the gradient or first derivative of an image, based on the hypothesis that rapid changes indicate sharp edges in the image, and thus are indicative of a lower degree of blurriness. Blur detection may be performed using Wavelet-based operator, which takes advantage of the capability of coefficients of the discrete wavelet transform to describe the frequency and spatial content of images. Blur detection may be performed using statistics-based operators take advantage of several image statistics as texture descriptors in order to compute a focus level. Blur detection may be performed by using discrete cosine transform (DCT) coefficients in order to compute a focus level of an image from its frequency content.

Continuing to refer to FIG. 3, computing device, processor, and/or module may be configured to precondition one or more training examples. For instance, and without limitation, where a machine learning model and/or process has one or more inputs and/or outputs requiring, transmitting, or receiving a certain number of bits, samples, or other units of data, one or more training examples' elements to be used as or compared to inputs and/or outputs may be modified to have such a number of units of data. For instance, a computing device, processor, and/or module may convert a smaller number of units, such as in a low pixel count image, into a desired number of units, for instance by upsampling and interpolating. As a non-limiting example, a low pixel count image may have 100 pixels, however a desired number of pixels may be 128. Processor may interpolate the low pixel count image to convert the 100 pixels into 128 pixels. It should also be noted that one of ordinary skill in the art, upon reading this disclosure, would know the various methods to interpolate a smaller number of data units such as samples, pixels, bits, or the like to a desired number of such units. In some instances, a set of interpolation rules may be trained by sets of highly detailed inputs and/or outputs and corresponding inputs and/or outputs downsampled to smaller numbers of units, and a neural network or other machine learning model that is trained to predict interpolated pixel values using the training data. As a non-limiting example, a sample input and/or output, such as a sample picture, with sample-expanded data units (e.g., pixels added between the original pixels) may be input to a neural network or machine-learning model and output a pseudo replica sample-picture with dummy values assigned to pixels between the original pixels based on a set of interpolation rules. As a non-limiting example, in the context of an image classifier, a machine-learning model may have a set of interpolation rules trained by sets of highly detailed images and images that have been downsampled to smaller numbers of pixels, and a neural network or other machine learning model that is trained using those examples to predict interpolated pixel values in a facial picture context. As a result, an input with sample-expanded data units (the ones added between the original data units, with dummy values) may be run through a trained neural network and/or model, which may fill in values to replace the dummy values. Alternatively or additionally, processor, computing device, and/or module may utilize sample expander methods, a low-pass filter, or both. As used in this disclosure, a “low-pass filter” is a filter that passes signals with a frequency lower than a selected cutoff frequency and attenuates signals with frequencies higher than the cutoff frequency. The exact frequency response of the filter depends on the filter design. Computing device, processor, and/or module may use averaging, such as luma or chroma averaging in images, to fill in data units in between original data units.

In some embodiments, and with continued reference to FIG. 3, computing device, processor, and/or module may down-sample elements of a training example to a desired lower number of data elements. As a non-limiting example, a high pixel count image may have 256 pixels, however a desired number of pixels may be 128. Processor may down-sample the high pixel count image to convert the 256 pixels into 128 pixels. In some embodiments, processor may be configured to perform downsampling on data. Downsampling, also known as decimation, may include removing every Nth entry in a sequence of samples, all but every Nth entry, or the like, which is a process known as “compression,” and may be performed, for instance by an N-sample compressor implemented using hardware or software. Anti-aliasing and/or anti-imaging filters, and/or low-pass filters, may be used to clean up side-effects of compression.

Further referring to FIG. 3, feature selection includes narrowing and/or filtering training data to exclude features and/or elements, or training data including such elements, that are not relevant to a purpose for which a trained machine-learning model and/or algorithm is being trained, and/or collection of features and/or elements, or training data including such elements, on the basis of relevance or utility for an intended task or purpose for a trained machine-learning model and/or algorithm is being trained. Feature selection may be implemented, without limitation, using any process described in this disclosure, including without limitation using training data classifiers, exclusion of outliers, or the like.

With continued reference to FIG. 3, feature scaling may include, without limitation, normalization of data entries, which may be accomplished by dividing numerical fields by norms thereof, for instance as performed for vector normalization. Feature scaling may include absolute maximum scaling, wherein each quantitative datum is divided by the maximum absolute value of all quantitative data of a set or subset of quantitative data. Feature scaling may include min-max scaling, in which each value X has a minimum value Xₘᵢₙ in a set or subset of values subtracted therefrom, with the result divided by the range of the values, give maximum value in the set or subset

$${X}_{max} : {X}_{new} = \frac{X - {X}_{min}}{{X}_{max} - {X}_{min}} .$$

Feature scaling may include mean normalization, which involves use of a mean value of a set and/or subset of values, Xₘₑₐₙ with maximum and minimum values:

$${X}_{new} = \frac{X - {X}_{mean}}{{X}_{max} - {X}_{min}} .$$

Feature scaling may include standardization, where a difference between X and Xₘₑₐₙ is divided by a standard deviation σ of a set or subset of values:

$${X}_{new} = \frac{X - {X}_{mean}}{σ} .$$

Scaling may be performed using a median value of a set or subset X_{median} and/or interquartile range (IQR), which represents the difference between the 25^{th} percentile value and the 50^{th} percentile value (or closest values thereto by a rounding protocol), such as:

$${X}_{new} = \frac{X - {X}_{median}}{IQR} .$$

Persons skilled in the art, upon reviewing the entirety of this disclosure, will be aware of various alternative or additional approaches that may be used for feature scaling.

Further referring to FIG. 3, computing device, processor, and/or module may be configured to perform one or more processes of data augmentation. “Data augmentation” as used in this disclosure is addition of data to a training set using elements and/or entries already in the dataset. Data augmentation may be accomplished, without limitation, using interpolation, generation of modified copies of existing entries and/or examples, and/or one or more generative AI processes, for instance using deep neural networks and/or generative adversarial networks; generative processes may be referred to alternatively in this context as “data synthesis” and as creating “synthetic data.” Augmentation may include performing one or more transformations on data, such as geometric, color space, affine, brightness, cropping, and/or contrast transformations of images.

Still referring to FIG. 3, machine-learning module 300 may be configured to perform a lazy-learning process 320 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 304. Heuristic may include selecting some number of highest-ranking associations and/or training data 304 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 3, machine-learning processes as described in this disclosure may be used to generate machine-learning models 324. A “machine-learning model,” as used in this disclosure, is a data structure representing and/or instantiating a mathematical and/or algorithmic representation of a relationship between inputs and outputs, as generated using any machine-learning process including without limitation any process as described above, and stored in memory; an input is submitted to a machine-learning model 324 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 324 may be generated by creating an artificial neural network, such as a convolutional neural network comprising an input layer of nodes, one or more intermediate layers, and an output layer of nodes. Connections between nodes may be created via the process of “training” the network, in which elements from a training data 304 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 3, machine-learning algorithms may include at least a supervised machine-learning process 328. At least a supervised machine-learning process 328, as defined herein, include algorithms that receive a training set relating a number of inputs to a number of outputs, and seek to generate one or more data structures representing and/or instantiating one or more mathematical relations relating inputs to outputs, where each of the one or more mathematical relations is optimal according to some criterion specified to the algorithm using some scoring function. For instance, a supervised learning algorithm may include biological extraction 116, user data 106, vocation data 118, directional inquiry 108, stress level datum 124, nutrient program 128, and the like as described above as inputs, stress level datum 124, nutrient program 128, outcome datum, guided recommendation, and the like as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 304. Persons skilled in the art, upon reviewing the entirety of this disclosure, will be aware of various possible variations of at least a supervised machine-learning process 328 that may be used to determine relation between inputs and outputs. Supervised machine-learning processes may include classification algorithms as defined above.

With further reference to FIG. 3, training a supervised machine-learning process may include, without limitation, iteratively updating coefficients, biases, weights based on an error function, expected loss, and/or risk function. For instance, an output generated by a supervised machine-learning model using an input example in a training example may be compared to an output example from the training example; an error function may be generated based on the comparison, which may include any error function suitable for use with any machine-learning algorithm described in this disclosure, including a square of a difference between one or more sets of compared values or the like. Such an error function may be used in turn to update one or more weights, biases, coefficients, or other parameters of a machine-learning model through any suitable process including without limitation gradient descent processes, least-squares processes, and/or other processes described in this disclosure. This may be done iteratively and/or recursively to gradually tune such weights, biases, coefficients, or other parameters. Updating may be performed, in neural networks, using one or more back-propagation algorithms. Iterative and/or recursive updates to weights, biases, coefficients, or other parameters as described above may be performed until currently available training data is exhausted and/or until a convergence test is passed, where a “convergence test” is a test for a condition selected as indicating that a model and/or weights, biases, coefficients, or other parameters thereof has reached a degree of accuracy. A convergence test may, for instance, compare a difference between two or more successive errors or error function values, where differences below a threshold amount may be taken to indicate convergence. Alternatively or additionally, one or more errors and/or error function values evaluated in training iterations may be compared to a threshold.

Still referring to FIG. 3, a computing device, processor, and/or module may be configured to perform method, method step, sequence of method steps and/or algorithm described in reference to this figure, in any order and with any degree of repetition. For instance, a computing device, processor, and/or module may be configured to perform a single step, sequence and/or algorithm repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. A computing device, processor, and/or module may perform any step, sequence of steps, or algorithm in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Further referring to FIG. 3, machine learning processes may include at least an unsupervised machine-learning processes 332. An unsupervised machine-learning process, as used herein, is a process that derives inferences in datasets without regard to labels; as a result, an unsupervised machine-learning process may be free to discover any structure, relationship, and/or correlation provided in the data. Unsupervised processes 332 may not require a response variable; unsupervised processes 332 may be used to find interesting patterns and/or inferences between variables, to determine a degree of correlation between two or more variables, or the like.

Still referring to FIG. 3, machine-learning module 300 may be designed and configured to create a machine-learning model 324 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task clastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 3, machine-learning algorithms may include, without limitation, linear discriminant analysis. Machine-learning algorithm may include quadratic discriminant analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized trees, AdaBoost, gradient tree boosting, and/or voting classifier methods. Machine-learning algorithms may include neural net algorithms, including convolutional neural net processes.

Still referring to FIG. 3, a machine-learning model and/or process may be deployed or instantiated by incorporation into a program, apparatus, system and/or module. For instance, and without limitation, a machine-learning model, neural network, and/or some or all parameters thereof may be stored and/or deployed in any memory or circuitry. Parameters such as coefficients, weights, and/or biases may be stored as circuit-based constants, such as arrays of wires and/or binary inputs and/or outputs set at logic “1” and “0” voltage levels in a logic circuit to represent a number according to any suitable encoding system including twos complement or the like or may be stored in any volatile and/or non-volatile memory. Similarly, mathematical operations and input and/or output of data to or from models, neural network layers, or the like may be instantiated in hardware circuitry and/or in the form of instructions in firmware, machine-code such as binary operation code instructions, assembly language, or any higher-order programming language. Any technology for hardware and/or software instantiation of memory, instructions, data structures, and/or algorithms may be used to instantiate a machine-learning process and/or model, including without limitation any combination of production and/or configuration of non-reconfigurable hardware elements, circuits, and/or modules such as without limitation ASICs, production and/or configuration of reconfigurable hardware elements, circuits, and/or modules such as without limitation FPGAs, production and/or of non-reconfigurable and/or configuration non-rewritable memory elements, circuits, and/or modules such as without limitation non-rewritable ROM, production and/or configuration of reconfigurable and/or rewritable memory elements, circuits, and/or modules such as without limitation rewritable ROM or other memory technology described in this disclosure, and/or production and/or configuration of any computing device and/or component thereof as described in this disclosure. Such deployed and/or instantiated machine-learning model and/or algorithm may receive inputs from any other process, module, and/or component described in this disclosure, and produce outputs to any other process, module, and/or component described in this disclosure.

Continuing to refer to FIG. 3, any process of training, retraining, deployment, and/or instantiation of any machine-learning model and/or algorithm may be performed and/or repeated after an initial deployment and/or instantiation to correct, refine, and/or improve the machine-learning model and/or algorithm. Such retraining, deployment, and/or instantiation may be performed as a periodic or regular process, such as retraining, deployment, and/or instantiation at regular elapsed time periods, after some measure of volume such as a number of bytes or other measures of data processed, a number of uses or performances of processes described in this disclosure, or the like, and/or according to a software, firmware, or other update schedule. Alternatively or additionally, retraining, deployment, and/or instantiation may be event-based, and may be triggered, without limitation, by user inputs indicating sub-optimal or otherwise problematic performance and/or by automated field testing and/or auditing processes, which may compare outputs of machine-learning models and/or algorithms, and/or errors and/or error functions thereof, to any thresholds, convergence tests, or the like, and/or may compare outputs of processes described herein to similar thresholds, convergence tests or the like. Event-based retraining, deployment, and/or instantiation may alternatively or additionally be triggered by receipt and/or generation of one or more new training examples; a number of new training examples may be compared to a preconfigured threshold, where exceeding the preconfigured threshold may trigger retraining, deployment, and/or instantiation.

Still referring to FIG. 3, retraining and/or additional training may be performed using any process for training described above, using any currently or previously deployed version of a machine-learning model and/or algorithm as a starting point. Training data for retraining may be collected, preconditioned, sorted, classified, sanitized or otherwise processed according to any process described in this disclosure. Training data may include, without limitation, training examples including inputs and correlated outputs used, received, and/or generated from any version of any system, module, machine-learning model or algorithm, apparatus, and/or method described in this disclosure; such examples may be modified and/or labeled according to user feedback or other processes to indicate desired results, and/or may have actual or measured results from a process being modeled and/or predicted by system, module, machine-learning model or algorithm, apparatus, and/or method as “desired” results to be compared to outputs for training processes as described above.

Redeployment may be performed using any reconfiguring and/or rewriting of reconfigurable and/or rewritable circuit and/or memory elements; alternatively, redeployment may be performed by production of new hardware and/or software components, circuits, instructions, or the like, which may be added to and/or may replace existing hardware and/or software components, circuits, instructions, or the like.

Further referring to FIG. 3, one or more processes or algorithms described above may be performed by at least a dedicated hardware unit 336. A “dedicated hardware unit,” for the purposes of this figure, is a hardware component, circuit, or the like, aside from a principal control circuit and/or processor performing method steps as described in this disclosure, that is specifically designated or selected to perform one or more specific tasks and/or processes described in reference to this figure, such as without limitation preconditioning and/or sanitization of training data and/or training a machine-learning algorithm and/or model. A dedicated hardware unit 336 may include, without limitation, a hardware unit that can perform iterative or massed calculations, such as matrix-based calculations to update or tune parameters, weights, coefficients, and/or biases of machine-learning models and/or neural networks, efficiently using pipelining, parallel processing, or the like; such a hardware unit may be optimized for such processes by, for instance, including dedicated circuitry for matrix and/or signal processing operations that includes, e.g., multiple arithmetic and/or logical circuit units such as multipliers and/or adders that can act simultaneously and/or in parallel or the like. Such dedicated hardware units 336 may include, without limitation, graphical processing units (GPUs), dedicated signal processing modules, FPGA or other reconfigurable hardware that has been configured to instantiate parallel processing units for one or more specific tasks, or the like, A computing device, processor, apparatus, or module may be configured to instruct one or more dedicated hardware units 336 to perform one or more operations described herein, such as evaluation of model and/or algorithm outputs, one-time or iterative updates to parameters, coefficients, weights, and/or biases, and/or any other operations such as vector and/or matrix operations as described in this disclosure.

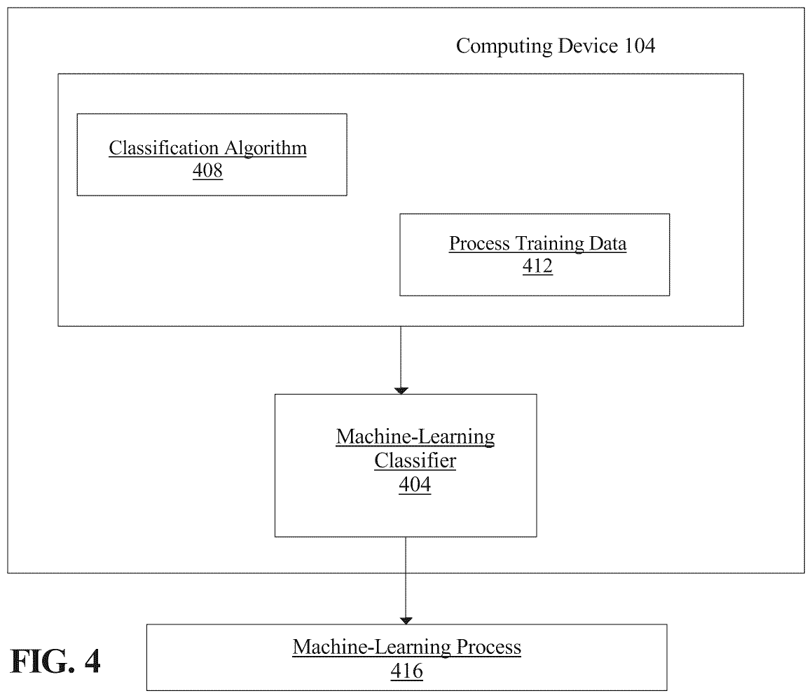

Referring now to FIG. 4, in an embodiment, computing device 104 may be configured to generate a directional response 132 where the computing device 104 is configured to generate a machine-learning classifier 404 as a function of a classification algorithm 408 and process training data 412. The process training data 412 correlates biological extraction to identifiers of a machine-learning process 416. The machine-learning process 416 is then outputted as a function of the biological extraction and the machine-learning classifier 404. As an example, a user suffering from Parkinson's disease may seek a directional response as to what type of job may be possible for someone with Parkinson's symptoms. Once the machine-learning classifier is trained, a machine-learning process 416 with a k-neighbor algorithm may be outputted which may provide the best accuracy using a biological extraction that incorporates Parkinson's disease. Other machine-learning processes may include, but are not limited to decision tree, linear discriminant analysis, support vector machine, and the like.

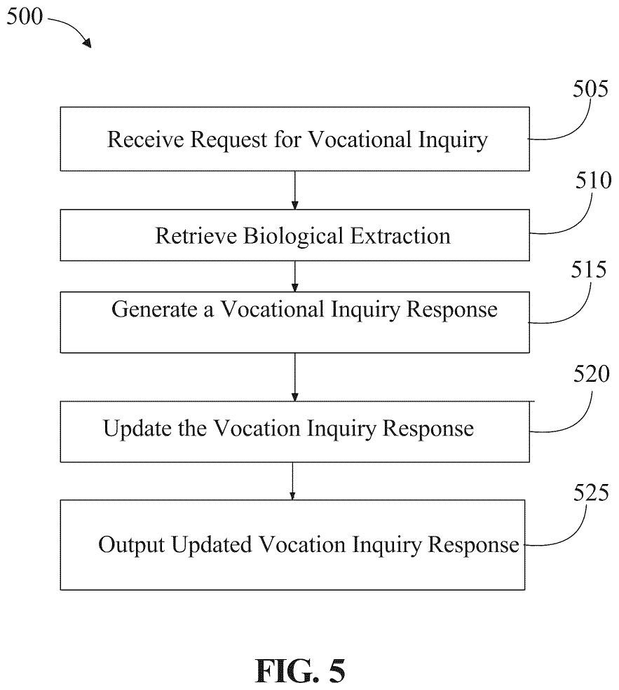

Now with reference to FIG. 5, a method 500 for generating a direction inquiry response is disclosed. At step 505, computing device may receive a request for a directional inquiry from a client device of a user. This may be implemented, without limitation, as described in FIGS. 1-4

Still referring to FIG. 5, at step 510, computing device retrieves a biological extraction from the user. This may be implemented, without limitation, as described in FIGS. 1-4. Physiological state data may include, without limitation any mental state data which may be implemented, without limitation, as described in FIGS. 1-4. A biological extraction may include a generic sequence. Retrieval of biological extraction may include, without limitation, reception of biological extraction from another computing device such as a device operated by a medical and/or diagnostic professional and/or entity, a device operated by the user, and/or any device suitable for use as a third-party device. This may be implemented, without limitation, as described in FIGS. 1-4.

Still referring to FIG. 5, at step 515, computing device may be configured to generate a directional response. A machine-learning process may be trained using directional training data correlating a plurality of biological extractions to a plurality of directions. Computing device may be configured to output the directional response as a function of the biological extraction from the user and the machine-learning process. This may be implemented, without limitation, as described in FIGS. 1-4. Computing device may generate using tendency training data correlating mental state data with tendency data and a second machine-learning process, a tendency model. This may be implemented, without limitation, as described in FIGS. 1-4. Computing device may be configured to generate a priority value as a function of the directional response and the tendency model. This may be implemented, without limitation, as described in FIGS. 1-4. Computing device may be configured to generate a directional response where the computing device is configured to generate a machine-learning classifier as a function of a classification algorithm and process training data. The process training data correlates biological extraction to identifiers of machine-learning processes. The machine-learning process is then outputted as a function of the biological extraction and the machine-learning classifier. This may be implemented, without limitation, as described in FIGS. 1-4.

At step 520, and still referring to FIG. 5, computing device may be configured to update the directional response as a function of the preferences of the user. This may be implemented, without limitation, as described in FIGS. 1-4. Computing device may update the directional response as a function of the type of work experience. This may be implemented, without limitation, as described in FIGS. 1-4.

Still referring to FIG. 5, at step 525, computing device may be configured to output the updated directional response to the device of user. This may be implemented, without limitation, as described in FIGS. 1-4. In an embodiment, the output transmitted to the user includes a plurality of hyperlinks as a function of the updated directional response. This may be implemented, without limitation, as described in FIGS. 1-4.

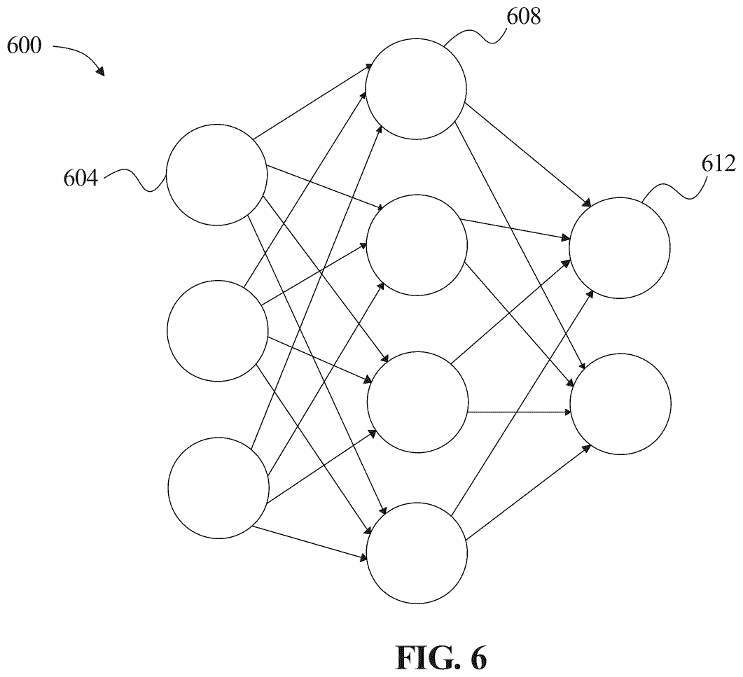

Referring now to FIG. 6, an exemplary embodiment of neural network 600 is illustrated. A neural network 600 also known as an artificial neural network, is a network of “nodes,” or data structures having one or more inputs, one or more outputs, and a function determining outputs based on inputs. Such nodes may be organized in a network, such as without limitation a convolutional neural network, including an input layer of nodes 604, one or more intermediate layers 608, and an output layer of nodes 612. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. Connections may run solely from input nodes toward output nodes in a “feed-forward” network, or may feed outputs of one layer back to inputs of the same or a different layer in a “recurrent network.” As a further non-limiting example, a neural network may include a convolutional neural network comprising an input layer of nodes, one or more intermediate layers, and an output layer of nodes. A “convolutional neural network,” as used in this disclosure, is a neural network in which at least one hidden layer is a convolutional layer that convolves inputs to that layer with a subset of inputs known as a “kernel,” along with one or more additional layers such as pooling layers, fully connected layers, and the like.

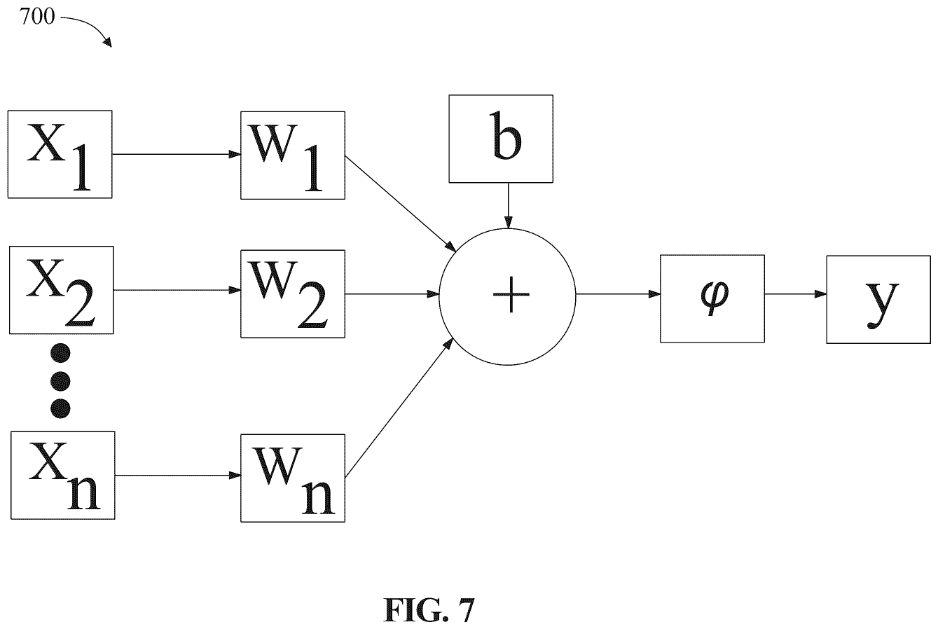

Referring now to FIG. 7, an exemplary embodiment of a node 700 of a neural network is illustrated. A node may include, without limitation, a plurality of inputs x; that may receive numerical values from inputs to a neural network containing the node and/or from other nodes. Node may perform one or more activation functions to produce its output given one or more inputs, such as without limitation computing a binary step function comparing an input to a threshold value and outputting either a logic 1 or logic 0 output or something equivalent, a linear activation function whereby an output is directly proportional to the input, and/or a non-linear activation function, wherein the output is not proportional to the input. Non-linear activation functions may include, without limitation, a sigmoid function of the form

$$f ( x ) = \frac{1}{1 - {e}^{- x}}$$

given input x, a tanh (hyperbolic tangent) function, of the form

$$\frac{{e}^{x} - {e}^{- x}}{{e}^{x} + {e}^{- x}} ,$$

a tanh derivative function such as f(x)=tanh²(x), a rectified linear unit function such as f(x)=max(0,x), a “leaky” and/or “parametric” rectified linear unit function such as f(x)=max(ax,x) for some a, an exponential linear units function such as

$$f ( x ) = \{ \begin{matrix}x for x \geq 0 \\ α ( {e}^{x} - 1 ) for x < 0\end{matrix}$$

for some value of α (this function may be replaced and/or weighted by its own derivative in some embodiments), a softmax function such as

$$f ( {x}_{i} ) = \frac{{e}^{x}}{{\sum }_{i} {x}_{i}}$$

where the inputs to an instant layer are xᵢ, a swish function such as f(x)=x*sigmoid(x), a Gaussian error linear unit function such as f (x)=a(1+tanh(\sqrt{2/π}(x+bx^{r}))) for some values of a, b, and r, and/or a scaled exponential linear unit function such as

$$f ( x ) = λ \{ \begin{matrix}α ( {e}^{x} - 1 ) for x < 0 \\ x for x \geq 0\end{matrix} .$$

Fundamentally, there is no limit to the nature of functions of inputs xᵢ that may be used as activation functions. As a non-limiting and illustrative example, node may perform a weighted sum of inputs using weights w; that are multiplied by respective inputs xᵢ. Additionally or alternatively, a bias b may be added to the weighted sum of the inputs such that an offset is added to each unit in the neural network layer that is independent of the input to the layer. The weighted sum may then be input into a function φ, which may generate one or more outputs y. Weight wᵢ applied to an input xᵢ may indicate whether the input is “excitatory,” indicating that it has strong influence on the one or more outputs y, for instance by the corresponding weight having a large numerical value, and/or a “inhibitory,” indicating it has a weak effect influence on the one more inputs y, for instance by the corresponding weight having a small numerical value. The values of weights wᵢ may be determined by training a neural network using training data, which may be performed using any suitable process as described above.

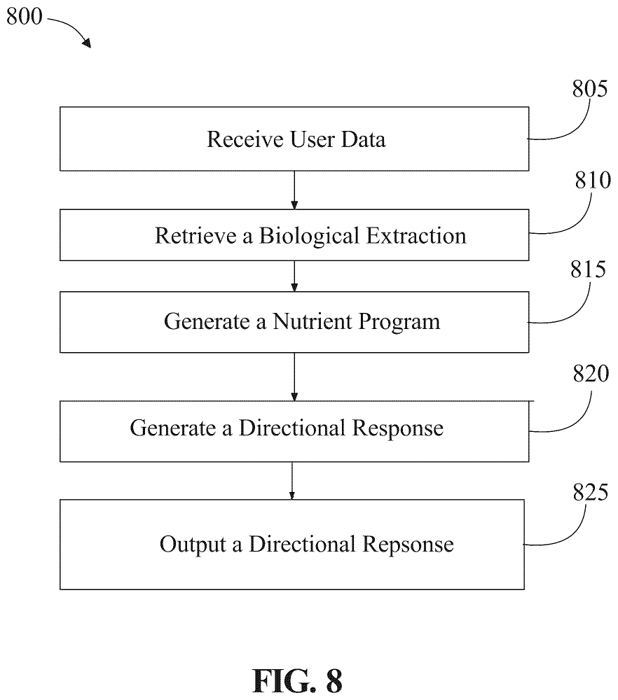

Referring now to FIG. 8, a flow diagram of an exemplary method 800 for generating a directional response using machine learning is illustrated. The method 800 contains a step 805 of receiving, using a computing device, user data, wherein the user data includes vocation data. In some embodiments, the user data may include information related to a family history of the user related to the biological extraction. These may be implemented as referenced to FIGS. 1-7.

With continued reference to FIG. 8, method 800 contains a step 810 of retrieving, using a computing device, a biological extraction of a user. In some embodiments, retrieving the biological extraction may include analyzing a food intake of the user to generate microbiome data of the biological extraction. These may be implemented as referenced to FIGS. 1-7.

With continued reference to FIG. 8, method 800 contains a step 815 of generating, using a computing device, a nutrient program as a function of vocation data, wherein generating the nutrient program includes generating program training data, wherein the program training data includes correlations between exemplary user data, exemplary biological extractions and exemplary nutrient programs, training a program machine-learning model using the program training data and generating the nutrient program using the trained program machine-learning model. These may be implemented as referenced to FIGS. 1-7.

With continued reference to FIG. 8, method 800 contains a step 820 of generating, using a computing device, a directional response as a function of a nutrient program. In some embodiments, generating the nutrient program may include generating the nutrient program as a function of the stress level datum. In some embodiments, method 800 may further include pairing, using the computing device, a third-party with the user as a function of the stress level datum. In some embodiments, method 800 may further include determining, using the computing device, an outcome datum related to the nutrient program and generating, using the computing device, the directional response as a function of the outcome datum. These may be implemented as referenced to FIGS. 1-7.

With continued reference to FIG. 8, method 800 contains a step 825 of outputting, using a computing device, a directional response. In some embodiments, method 800 may further include determining, using the computing device, a stress level datum as a function of the biological extraction. In some embodiments, determining the stress level datum may include extracting at least a keyword from the biological extraction using a language processing module and determining the stress level datum as a function of the at least a keyword. In some embodiments, determining the stress level datum may include generating stress level training data, wherein the stress level training data comprises correlations between exemplary biological extractions and exemplary stress level datums, training a stress level machine-learning model using the stress level training data and determining the stress level using the trained stress level machine-learning model. In some embodiments, method 800 may further include generating, using the computing device, a tendency model, generating, using the computing device, at least one priority value as a function of the directional response and the tendency model and removing, using the computing device, a priority value of the at least one priority value as a function of a filter comprising a user-selected threshold value for the at least one priority value. These may be implemented as referenced to FIGS. 1-7.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

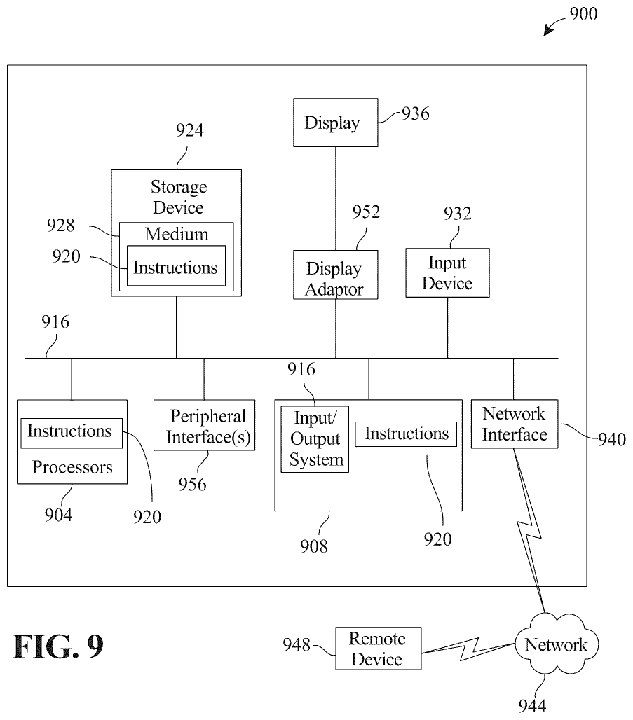

FIG. 9 shows a diagrammatic representation of one embodiment of a computing device in the exemplary form of a computer system 900 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 900 includes a processor 904 and a memory 908 that communicate with each other, and with other components, via a bus 912. Bus 912 may include any of several types of bus structures including, but not limited to, a memory bus, a memory controller, a peripheral bus, a local bus, and any combinations thereof, using any of a variety of bus architectures.

Processor 904 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 904 may be organized according to Von Neumann and/or Harvard architecture as a non-limiting example. Processor 904 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC)

Memory 908 may include various components (e.g., machine-readable media) including, but not limited to, a random-access memory component, a read only component, and any combinations thereof. In one example, a basic input/output system 916 (BIOS), including basic routines that help to transfer information between elements within computer system 900, such as during start-up, may be stored in memory 908. Memory 908 may also include (e.g., stored on one or more machine-readable media) instructions (e.g., software) 920 embodying any one or more of the aspects and/or methodologies of the present disclosure. In another example, memory 908 may further include any number of program modules including, but not limited to, an operating system, one or more application programs, other program modules, program data, and any combinations thereof.

Computer system 900 may also include a storage device 924. Examples of a storage device (e.g., storage device 924) include, but are not limited to, a hard disk drive, a magnetic disk drive, an optical disc drive in combination with an optical medium, a solid-state memory device, and any combinations thereof. Storage device 924 may be connected to bus 912 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 924 (or one or more components thereof) may be removably interfaced with computer system 900 (e.g., via an external port connector (not shown)). Particularly, storage device 924 and an associated machine-readable medium 928 may provide nonvolatile and/or volatile storage of machine-readable instructions, data structures, program modules, and/or other data for computer system 900. In one example, software 920 may reside, completely or partially, within machine-readable medium 928. In another example, software 920 may reside, completely or partially, within processor 904.

Computer system 900 may also include an input device 932. In one example, a user of computer system 900 may enter commands and/or other information into computer system 900 via input device 932. Examples of an input device 932 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 932 may be interfaced to bus 912 via any of a variety of interfaces (not shown) including, but not limited to, a serial interface, a parallel interface, a game port, a USB interface, a FIREWIRE interface, a direct interface to bus 912, and any combinations thereof. Input device 932 may include a touch screen interface that may be a part of or separate from display 936, discussed further below. Input device 932 may be utilized as a user selection device for selecting one or more graphical representations in a graphical interface as described above.

A user may also input commands and/or other information to computer system 900 via storage device 924 (e.g., a removable disk drive, a flash drive, etc.) and/or network interface device 940. A network interface device, such as network interface device 940, may be utilized for connecting computer system 900 to one or more of a variety of networks, such as network 944, and one or more remote devices 948 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 944, may employ a wired and/or a wireless mode of communication. In general, any network topology may be used. Information (e.g., data, software 920, etc.) may be communicated to and/or from computer system 900 via network interface device 940.

Computer system 900 may further include a video display adapter 952 for communicating a displayable image to a display device, such as display device 936. Examples of a display device include, but are not limited to, a liquid crystal display (LCD), a cathode ray tube (CRT), a plasma display, a light emitting diode (LED) display, and any combinations thereof. Display adapter 952 and display device 936 may be utilized in combination with processor 904 to provide graphical representations of aspects of the present disclosure. In addition to a display device, computer system 900 may include one or more other peripheral output devices including, but not limited to, an audio speaker, a printer, and any combinations thereof. Such peripheral output devices may be connected to bus 912 via a peripheral interface 956. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A system for generating a directional response using machine learning, the system comprising:
a computing device, wherein the computing device is configured to:
receive user data;
retrieve a biological extraction of a user;
generate a nutrient program as a function of the user data, wherein generating the nutrient program comprises:
generating program training data, wherein the program training data comprises exemplary user data and exemplary biological extractions correlated to exemplary nutrient programs;
training a program machine-learning model using the program training data; and
generating the nutrient program using the trained program machine-learning model;
generate a directional response as a function of the nutrient program; and
output the directional response.

2. The system of claim 1, wherein the user data comprises information related to a family history of the user related to the biological extraction.

3. The system of claim 1, wherein retrieving the biological extraction comprises analyzing a food intake of the user to generate microbiome data of the biological extraction.

4. The system of claim 1, wherein the computing device is further configured to determine a stress level datum as a function of the biological extraction.

5. The system of claim 4, wherein determining the stress level datum comprises:
extracting at least a keyword from the biological extraction using a language processing module; and
determining the stress level datum as a function of the at least a keyword.

6. The system of claim 4, wherein determining the stress level datum comprises:
generating stress level training data, wherein the stress level training data comprises exemplary biological extractions correlated to exemplary stress level datums;
training a stress level machine-learning model using the stress level training data; and
determining the stress level using the trained stress level machine-learning model.

7. The system of claim 4, wherein generating the nutrient program comprises generating the nutrient program as a function of the stress level datum.

8. The system of claim 4, wherein the computing device is further configured to pair a third-party with the user as a function of the stress level datum and user data comprising vocation data.

9. The system of claim 1, wherein the computing device is further configured to:
determine an outcome datum related to the nutrient program; and
generate the directional response as a function of the outcome datum.

10. The system of claim 1, wherein the computing device is further configured to:
generate a tendency model;
generate at least one priority value as a function of the directional response and the tendency model; and
remove a priority value of the at least one priority value as a function of a filter comprising a user-selected threshold value for the at least one priority value.

11. A method for generating a directional response using machine learning, the method comprising:
receiving, using a computing device, user data;
retrieving, using the computing device, a biological extraction of a user;
generating, using the computing device, a nutrient program as a function of the user data, wherein generating the nutrient program comprises:
generating program training data, wherein the program training data comprises exemplary user data and exemplary biological extractions correlated to exemplary nutrient programs;
training a program machine-learning model using the program training data; and
generating the nutrient program using the trained program machine-learning model;
generating, using the computing device, a directional response as a function of the nutrient program; and
outputting, using the computing device, the directional response.

12. The method of claim 11, wherein the user data comprises information related to a family history of the user related to the biological extraction.

13. The method of claim 11, wherein retrieving the biological extraction comprises analyzing a food intake of the user to generate microbiome data of the biological extraction.

14. The method of claim 11, further comprising:
determining, using the computing device, a stress level datum as a function of the biological extraction.

15. The method of claim 14, wherein determining the stress level datum comprises:
extracting at least a keyword from the biological extraction using a language processing module; and
determining the stress level datum as a function of the at least a keyword.

16. The method of claim 14, wherein determining the stress level datum comprises:
generating stress level training data, wherein the stress level training data comprises exemplary biological extractions correlated to exemplary stress level datums;
training a stress level machine-learning model using the stress level training data; and
determining the stress level using the trained stress level machine-learning model.

17. The method of claim 14, wherein generating the nutrient program comprises generating the nutrient program as a function of the stress level datum.

18. The method of claim 14, further comprising:
pairing, using the computing device, a third-party with the user as a function of the stress level datum and user data comprising vocation data.

19. The method of claim 11, further comprising:
determining, using the computing device, an outcome datum related to the nutrient program; and
generating, using the computing device, the directional response as a function of the outcome datum.

20. The method of claim 11, further comprising:
generating, using the computing device, a tendency model;
generating, using the computing device, at least one priority value as a function of the directional response and the tendency model; and
removing, using the computing device, a priority value of the at least one priority value as a function of a filter comprising a user-selected threshold value for the at least one priority value.
